(19) United States
(12) Patent Application Publication
(10) Pub. No.: US 2008/0208230 A1
(43) Pub. Date: Aug. 28, 2008

(54) EXPANDABLE ROTATING DEVICE AND METHOD FOR TISSUE ASPIRATION

(76) Inventors: Singfatt CHIN, Pleasanton, CA (US); Daniel H. Kim, Houston, TX (US); John T. To, Newark, CA (US)

Correspondence Address: MORRISON & FOERSTER LLP, 755 PAGE MILL RD, PALO ALTO, CA, 94304-1018, US

(21) Appl. No.: 12/035,323

(22) Filed: Feb. 21, 2008

Related U.S. Application Data
(60) Provisional application No. 60/891,177, filed on Feb. 22, 2007.

(51) Int. Cl. A61B 17/32; C21D 1/30

(52) U.S. Cl. 606/167; 148/559

(57) ABSTRACT

An apparatus and method for removing tissue and/or other material from a patient includes a shaft and a tissue disrupting mechanism operatively coupled to the shaft. The shaft may be coupled to a handpiece or a robotic or remote-controlled system. The mechanism may comprise a rotatable or other movable element having a distal portion with fixed or adjustable radial dimensions. The mechanism may have one or more tissue cutting, chopping, grinding, emulsifying or disrupting features with an adjustable outer diameter for removing substantial tissues. The apparatus may be configured to urge or draw substantial material into the device upon rotation or other movement of the shaft and/or tissue, and may optionally be coupled to sources of suction or aspiration. A radiofrequency or other energy source is optionally included for tissue ablation or other tissue remodeling effects, and/or to enhance coagulation.

### CROSS-REFERENCE TO RELATED APPLICATIONS

This application claims priority under 35 U.S.C. §119(e) to U.S. Provisional Patent Application No. 60/891,177, filed Feb. 22, 2007, the disclosure of which is hereby incorporated by reference in its entirety.

### BACKGROUND

It is sometimes desirable to remove a portion of tissue from humans and other animals, particularly in the diagnosis and/or treatment of patients with herniated disc or other spinal disorders, cancerous tumors, pre-malignant conditions, benign prostatic hyperplasia (BPH) or prostatic cancer, liver disease, breast disease including cancer, brain disease including cancer, and other diseases or disorders at any location in a patient.

For example, the spinal column includes, among other structures, the bony vertebrae, which surround the spinal cord, and the intervertebral discs. Each vertebra is separated by an intervertebral disc, which comprises an outer membrane ring called the annulus fibrosus and the inner filling area known as the nucleus pulposus. The fibrous makeup of the disc provides tensile strength, thus providing functional significance to the well-being of the body. In a healthy spine, the discs maintain separation between the vertebrae, promote fluid circulation throughout the spine, and provide a cushioning effect between the bony vertebral structures.

Due to the elastic nature of an intervertebral disc, the disc may be subject to injury if the disc becomes overstressed, for example, by trauma to the spine, excess body weight, improper mechanical movements and the like. Intervertebral disc injuries and other abnormalities may result in serious back pain and physical disability, and may become chronic and difficult to treat. Disc abnormalities include, but are not limited to, localized tears or fissures in the disc annulus, localized disc herniations and circumferential bulging discs. Discs may also experience further degeneration over time which can accelerate these problems.

Disc fissures may result from structural degeneration of fibrous components of the disc annulus (annulus fibrosis). More specifically, fibrous components of the annulus may become separated in particular areas, creating a fissure within the annulus. Sometimes the fissure is accompanied by extrusion of material from the disc nucleus (nucleus pulposus) and into the fissure. Biochemicals and other bodily substances may escape from the disc, which may cause irritation to surrounding structures. These disc fissures are known to be extremely painful. The fissure may also be associated with herniation of that portion of the annulus wall.

Disc herniation is a type of disc degenerative disorder where the disc is either completely or partially broken, causing rupture and leaking of the nucleus material out onto surrounding nerves. Herniation also creates excess disc tissue that cannot be contained within the volume of the disc. This build-up creates added pressure within the spine, and may impact nearby structures. For example, the herniated disc may impinge on a nerve, causing considerable pain for a patient. Often this type of disorder can lead to radiating pain beyond the back, as well as numbness, weakness in the muscles, and loss of physical movement.

With a contained disc herniation, the nucleus pulposus may work its way partly through the annulus. The outward protrusion of fibrous and nuclear material can press upon the spinal nerves or irritate other body structures. Another common disc problem occurs when the entire disc bulges circumferentially about the annulus rather than at specific, isolated locations. This may occur over time, for example, when the disc weakens, bulges, and takes on a “roll” shape. The joint may become unstable and one vertebra may eventually settle on top of another vertebra. This problem may escalate as the body ages, and may account for a person's shortened stature in old age. Osteophytes may also form on the outer surface of the disc and further encroach upon the spinal canal and nerve foramina. This condition is called spondylosis.

Traditional non-surgical treatments of disc degeneration and abnormalities include bed rest, pain and muscle relaxant medications, physical therapy and steroid injections. Such therapies are directed primarily at pain relief and delaying further disc degeneration. In many cases, non-surgical approaches may fail and surgical methods of treatment may be considered. Pain treatment via analgesic or anti-inflammatory drugs is one approach for handling disc herniations, but since the impinged nerve and/or disc rupture still remains, surgical alternatives may be considered to treat the problem directly at its site by nerve decompression. A long-term solution may involve surgical removal of disc material such that the disc is reduced to a smaller volume and is no longer impinging on the nerve. This is possible with a discectomy surgery, where the herniated portion and ruptured material of the disc are removed, or a percutaneous discectomy where nuclear disc material is removed using a surgical cutting instrument.

Other surgical treatments include spinal fixation, which are methods aimed at causing the vertebrae above and below the injured disc to fuse together and to form a single piece of bone. This procedure may be carried out with or without discectomy (surgical removal of the disc). Another procedure, endoscopic discectomy, involves removing tissue from the disc percutaneously in order to reduce the volume of the disc, thereby reducing impingement of the surface of the disc on nearby nerves.

Notwithstanding the above, there still exists a need for devices and methods for safely, accurately and effectively removing material or tissue from the body.

### BRIEF SUMMARY

An apparatus and method for removing tissue and/or other material from a patient is provided. The apparatus generally includes a shaft and a tissue disrupting mechanism operatively coupled to the shaft. In some embodiments, the shaft is coupled to a handpiece, but in other embodiments, the shaft may be coupled to a robotic or remote-controlled system. The mechanism may comprise a rotatable or other movable element having a distal portion with fixed or adjustable radial dimensions. For example, the mechanism may have one or more tissue disrupting members with cutting, chopping, grinding, debriding or other disrupting features, and an adjustable outer diameter or other transverse dimension for removing substantial tissues. In some embodiments, the apparatus may be configured to urge or draw substantial material into the device upon rotation or other movement of the shaft and/or tissue, and may optionally include suction or aspiration mechanisms. A radiofrequency or other energy source is optionally included for tissue ablation or other tissue remodeling effects, and/or to enhance coagulation.

Some embodiments may be used to remove unwanted, diseased, or even healthy bodily materials for medical treatment and/or therapeutic purposes. Some embodiments may be suitable for use in various surgical settings and may be suitable for performing various minimally invasive material removal procedures. Minimally invasive or endoscopic procedures may involve introducing the apparatus into the body and removing the apparatus from the body. Some embodiments may be used for a range of different specific medical treatments, e.g., diagnostic and therapeutic purposes.

In one embodiment, a device for removing material from a body is provided, comprising a drive shaft comprising a proximal section, a distal section and a longitudinal shaft axis therebetween, a motor coupled to the proximal section of the drive shaft, and at least one tissue disrupting member comprising a proximal section and a distal section and having a collapsed configuration and a deployed configuration. The proximal section of the tissue disrupting member is coupled to the distal section of the drive shaft at a proximal coupling zone, and wherein the collapsed configuration of the tissue disrupting member exerts greater bending stress on the proximal end of the tissue disrupting member than the deployed configuration. The proximal section of the disrupting member may be integral with the distal section of the drive shaft. In some embodiments, the tissue disrupting member is preshaped to its deployed configuration. The deployed configuration of at least one tissue disrupting member may comprise a bend that is distal to the proximal coupling zone. In some embodiments, the bend is at least about 1 mm, 1.5 mm or 2 mm distal to the coupling zone. In some embodiments, the device comprises at least two tissue disrupting members and at least one slot between at least two tissue disrupting members, and wherein the at least one slot comprises a proximal slot end and a distal slot end. For example, the device may comprise about three tissue disrupting members to about six tissue disrupting members in some instances. Sometimes, the proximal slot end of at least one slot may be longitudinally located between the proximal coupling zone and the bend of at least one tissue disrupting member. The distal slot end of at least one slot may also be longitudinally located distal to the bend of at least one tissue disrupting member. Also, the tissue disrupting member proximal to the bend may comprise a generally straight configuration in some embodiments. The tissue disrupting member may be an elongate disrupting member, and the distal section of the elongate tissue disrupting member may be coupled to a slide member that is slidably located in a lumen of the drive shaft. In some examples, the distal end of at least one tissue disrupting member comprises a free distal end. In some embodiments, the device may further comprise a helical transport structure. The helical structure may be integral with a surface of the drive shaft, or may be independently movable from the drive shaft. In some embodiments, the device further comprises a housing with a motor cavity, a drive shaft aperture, a drive shaft lumen between the motor cavity and the drive shaft aperture, a tubing connector and a lumen between the drive shaft lumen and the tubing connector, and a motor controller. The motor controller may be configured to permit user-controlled movement of the drive shaft in two or more directions, and in some embodiments, the device may also further comprise a slide controller configured to permit user-controlled movement of the slide member with respect to the drive shaft. In some embodiments, at least one tissue disrupting member, if not all, may slidably reside in a distal lumen of the distal section of the drive shaft. In some embodiments, at least one tissue disrupting member comprises an elongate wire, polymer or fiber structure, while in other embodiments, at least one tissue disrupting member comprises a plate member. The plate member may be a non-planar plate member, and in some embodiments, the proximal end of the plate member comprises a flange configuration. In some embodiments, the outermost portion of the tissue disrupting member is located about 1 mm to about 5 mm from the longitudinal axis of the drive shaft in the collapsed configuration and about 2 mm to 13 mm in the deployed position. In some embodiments, at least one tissue disrupting member comprises a material selected from a group consisting of nickel-titanium alloy, stainless steel, cobalt-chromium alloy, nickel-cobalt-chromium-molybdenum alloy, and titanium-aluminum-vanadium alloy.

In another embodiment, a method of removing tissue is provided, comprising providing a tissue disrupting device comprising a drive shaft and a plurality of tissue disrupting members coupled to the drive shaft at a coupling zone, exerting a greater stress on the plurality of non-linear tissue disrupting members at a distal stress zone that is distal to the coupling zone and a lesser stress at a proximal stress zone located between the coupling zone and the distal stress zone to restrain the tissue disrupting device, inserting the restrained tissue disrupting device into a body, positioning the restrained tissue disrupting device about a target area in the body, reducing the greater stress at the distal stress zone of the plurality of non-linear tissue disrupting members, and actuating the plurality of tissue disrupting members to disrupt tissue at the target area. In some embodiments, actuating the plurality of tissue disrupting member comprises rotating the plurality of disrupting members at a speed of about 5,000 rpm to about 100,000 rpm, but in other embodiments, the speed may be about 3,000 rpm to about 20,000 rpm. In some embodiments, the method may further comprise rotating an auger to transport disrupted tissue away from the target area, and in further embodiments, the plurality of tissue disrupting members and the auger are rotated independently. The method optionally further comprise applying suction to the target area to transport disrupted tissue away from the target area and/or adjusting the greater stress at the distal stress zone to modify at least one dimension of the plurality of tissue disrupting members. Some embodiments may also further comprise adjusting the greater stress at the distal stress zone to reduce at least one dimension of the plurality of tissue disrupting members, repositioning the tissue disrupting device so that the plurality of tissue disrupting members to a second target area, readjusting the greater stress at the distal stress zone to increase at least one dimension of the plurality of tissue disrupting members, and rotating the strip portion to disrupt tissue at the second target area.

In another embodiment, a method of manufacturing a disrupting device is provided, comprising providing a tubular body comprising a proximal end, a distal end, and a midsection therebetween, creating a plurality of struts with disrupting edges in the midsection of the tubular body by forming a plurality of slots between the proximal and distal ends of the tubular body, shaping the midsection of the tubular body in a radially outward direction without straining the tubular body by more than 8%, heat annealing the tubular body to reduce the strain, reshaping the heat annealed midsection in a radially outward direction without straining the tubular body by more than 8%, and heat annealing the reshaped tubular body to reduce the strain. In some embodiments, the method may further comprise coupling the tubular body to a motor with a rotatable shaft.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures:

FIG. 1 is a side elevational view of an embodiment of a tissue disrupting apparatus;

FIG. 2 is a detailed cutaway view of the apparatus in FIG. 1;

FIGS. 3A and 3B are various views of one embodiment of a tissue disrupting element in partially retracted and fully retracted configurations, respectively;

FIGS. 4A to 4C are various elevational views of the tissue disrupting element in FIGS. 3A and 3B in an extended position; FIG. 4D is a cross-sectional view of the tissue disrupting element in FIGS. 4A to 4C;

FIGS. 5A and 5B are side elevational views of another embodiment of a tissue disrupting element;

FIG. 6A is a side elevational view of another embodiment of a tissue disrupting element in a reduced configuration; FIGS. 6B and 6C are side elevational views of the tissue disrupting element of FIG. 6A in an expanded configuration;

FIGS. 7A and 7B are perspective views of the tissue disrupting element in FIGS. 6A to 6C in its reduced and expanded configurations, respectively;

FIGS. 8A and 8B are perspective and side elevational views of another embodiment of a tissue disrupting apparatus; FIG. 8C is a component view of the tissue disrupting apparatus in FIGS. 8A to 8B; and FIG. 8D is a cross-sectional view of the tissue disrupting apparatus in 8A and 8B with a portion of the housing removed;

FIGS. 9A to 9C are various elevational views of another embodiment of a tissue disrupting element in a reduced configuration; FIGS. 9D to 9F are various elevational views of the tissue disrupting element of FIGS. 9A to 9C in an expanded configuration; FIG. 9G is a detailed view of the tissue disrupting element in FIG. 9D;

FIGS. 10A to 10D are various elevational views of one embodiment of a disrupter assembly in a reduced configuration;

FIGS. 11A to 11D are various elevational views of the disrupter assembly in FIGS. 10A to 10D in an expanded configuration;

FIG. 12 is a component view of an embodiment of a tissue disrupting element with the disrupter assembly of FIGS. 10A to 10D;

FIG. 13 is an elevational view of the tissue disrupting element of FIG. 12;

FIG. 14 is another component view of the tissue disrupting element in FIG. 12;

FIGS. 15 to 17 are schematic representations of various embodiments of a disrupter structure;

FIGS. 18A and 18B depict another embodiment of a tissue disrupting apparatus; FIGS. 18C and 18D are perspective and end views of one disrupting element, respectively; and

FIGS. 19 to 21 illustrate additional embodiments of disrupting elements.

### DETAILED DESCRIPTION OF THE INVENTION

The present invention and the objects and advantages thereof will be more clearly understood and appreciated with respect to the following Detailed Description, when considered in conjunction with the accompanying Drawings.

The removal of tissue or cells from a patient may be performed using a variety of outpatient and inpatient procedures and surgeries, both diagnostic and therapeutic. The purpose of the procedure and the amount of tissue to be removed may affect the selection of a particular procedure and the type of access used to reach the target tissue.

In some embodiments, the tissue disrupting apparatus comprises a tissue disrupting element that may be rotated, vibrated or reciprocated to remove at least a portion of the tissue or body structures contacting the tissue disrupting element. The tissue disrupting element may be coupled to a shaft that permits the tissue disrupting element to be inserted into a remote site of the body and controlled at a different site. The tissue disrupting element may disrupt tissue or body structures from the impact force or rotation speed of the tissue disrupting elements. In some embodiments, the tissue disrupting element may be further configured with a cutting edge or piercing member to enhance removal of tissue or other body matter.

FIG. 1 depicts one embodiment of a tissue disrupting apparatus 2, comprising an outer tube 4 coupled to a housing 6. Outer tube 4 is may be attached to a tissue disrupting element 8, which is described in greater detail below. Housing 6 contains one or more components configured to control tissue disrupting element 8 and other optional features of tissue disrupting apparatus 2. Tissue disrupting element 8, which will be described in greater detail below, may be configured to cut, chop, grind, burr, debride and/or emulsify tissue, for example. Emulsification includes, for example, forming a suspension of tissue particles in a medium. The medium may comprise existing liquid at the target site, liquid added through the tissue disrupting apparatus, and/or liquid generated by the disruption of the tissue. These optional components may include but are not limited to a motor configured to rotate or move the tissue disrupting element, a power source or power interface, a motor controller, a tissue transport assembly, an energy delivery or cryotherapy assembly, a therapeutic agent delivery assembly, a light source, and one or more fluid seals. The optional tissue transport assembly may comprise a suction assembly and/or a mechanical aspiration assembly. One or more of these components may act through outer tube 4 to manipulate the tissue disrupting element and/or other components located distal to housing 6, or from housing 6 directly. In FIG. 1, for example, tissue disrupting apparatus 2 further comprises an optional port 20 that may be attached to an aspiration or suction source to facilitate transport of tissue or fluid out of the target site or patient. The suction source may be a powered vacuum pump, a wall suction outlet, or a syringe. These and other components will be described in greater detail below.

In the specific embodiment depicted in FIG. 1, for example, housing 6 further comprises a control interface 10 that may be used to control the power state of tissue disrupting apparatus 2, including but not limited to on and off states. In this particular embodiment, control interface 10 comprises a lever or pivot member, but in other embodiments, control interface 10 may comprise a push button, a slide, a dial or knob. In some embodiments, control interface 10 may also adjust the motor speed and/or movement direction of tissue disrupting element 8. A bi-directional tissue disrupting apparatus may be provided as a potential safety feature should the tissue disrupting element 8 get lodged in a body tissue or structure. In some situations, dislodging may be achieved by reversing the direction of rotation. Control interface 10 may be analog or digital, and may comprise one or more detent positions to facilitate selection of one or more pre-selected settings. In other embodiments, a separate motor control interface may be provided for one or more features of the motor. In still other embodiments, control interfaces for other features of the tissue disrupting apparatus may be provided.

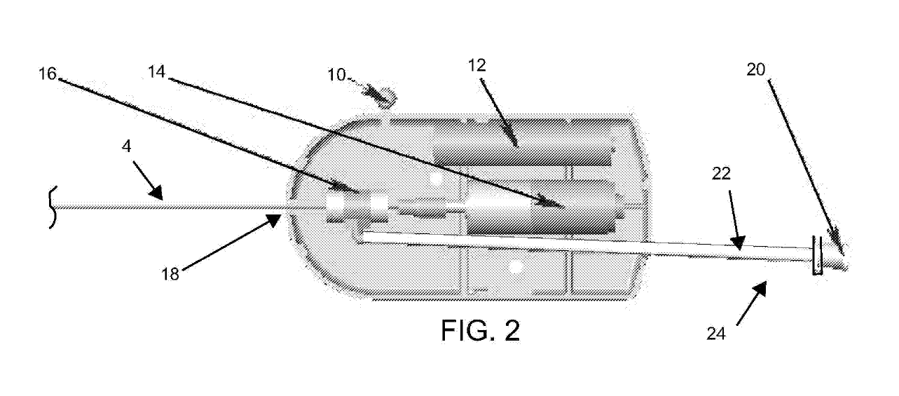

FIG. 2 depicts tissue disrupting apparatus 2 with a portion of housing 6 removed to show various internal components. For example, tissue disrupting apparatus 2 further comprises a battery 12 to provide power to the motor 14 which drives tissue disrupting element 8 via outer tube 4. In other embodiments, a connector to an external power source may be provided in addition to, or in lieu of, battery 12. The type of battery and power provided may differ depending upon the particular power needs of the motor and/or other components of tissue disrupting apparatus 2.

In some embodiments, motor 14 of tissue disrupting apparatus 2 is a DC motor, but in other embodiments, motor 14 may be configured with any of a variety of motors, including but not limited to an AC or a universal motor. Motor 14 may be a torque, brushed, brushless or coreless type of motor. In some embodiments, motor 14 may be configured to provide a rotational speed of about 500 rpm to about 200,000 rpm, sometimes about 1,000 rpm to about 40,000 rpm, and at other times about 5,000 rpm to about 20,000 rpm. Motor 14 may act on tissue disrupting element 8 via outer tube 4 or a drive member located within outer tube 4. In some further embodiments, a fluid seal 16 may be used to protect motor 14 and/or other components of housing 6 from any fluids or other materials that may be transported through outer tube 4, or through the housing aperture 18. In some embodiments, a connector or seal may be provided about housing aperture 18 to permit coupling of housing 6 to a trocar, an introducer, a cannula or other tubular member in which tissue disrupting element 8 and outer tube 4 are inserted. In some embodiments, the tissue disrupting apparatus may be used with an introducer or cannula comprising an outer diameter of about 0.01 cm to about 1.5 cm or more, sometimes about 0.1 cm to about 1 cm, and other times about 2 mm to about 6 mm.

As shown in FIGS. 1 and 2, some embodiments of tissue disrupting apparatus 2 further comprise a conduit 24 which may be used to connect tissue disrupting apparatus 2 and an aspiration or suction source. An aspiration or suction source may be used, for example, to transport fluid or material through a lumen of outer tube 4 or through a tubular member in which outer tube 4 is inserted. In one particular embodiment, conduit 24 comprises port 20 which communicates with fluid seal 16 via a length of tubing 22. Fluid seal 16 is configured to permit flow of fluid or material between outer tube 4 and tubing 22, while permitting movement of outer tube 4 or a drive member therein coupled to motor 14. In other embodiments, conduit 24 may further comprise additional components, including but not limited to a fluid or material trap, which may be located within or attached to housing 6, or attached to port 20 or tubing 22, or located anywhere else along the pathway from tissue disrupting element 8 to the suction source. In some embodiments, a separate port may be provided for infusing or injecting substances into target site using the tissue disrupting apparatus 2. In other embodiments, conduit 24 may be used for both withdrawal and infusion of materials or substances, or for infusion only. In other embodiments, a port may be used to insert coagulation catheter, an ablation catheter or other energy delivery device to the target site.

In some embodiments, outer tube 4 comprises an outer tubular member with at least one lumen, and an elongate drive member configured to mechanically couple the motor to tissue disrupting element 8. In other embodiments, outer tube 4 may contain additional members, for example, to adjust or control the configuration of tissue disrupting element 8. In some embodiments, outer tube 4 may comprise one or more lumens containing control wires, which may be used to manipulate the deflections of the distal end of outer tube 4. Outer tube 4 and optional drive members may be rigid or flexible. Outer tube 4 may be pre-shaped with a linear or a non-linear configuration. In some embodiments, outer tube 4 and the components therein may be designed to be user deformable, which may facilitate access to particular target sites, or may be steerable using a steering mechanism comprising one or more pull wires or tension elements. In some embodiments, a stiffening wire or element may be inserted into outer tube 4 to provide additional stiffness to tissue disrupting apparatus 2. The length of outer tube 4 between the tissue disrupting element and the motor may vary from about 0 cm to about 30 cm or more in some embodiments, sometimes about 4 cm to about 20 cm, and other times about 10 cm to about 14 cm.

In other embodiments, the tissue disrupting apparatus may comprise a tissue disrupting element that may be detachably attachable to the shaft of a motor or coupled to a motor. In still other embodiments, the tissue disrupting apparatus may comprise a tissue disrupting element coupled to a shaft, wherein the shaft may be detachably attachable to a motor or a shaft coupled to a motor.

In some embodiments, housing 6 is configured with a size and/or shape that permits handheld use of tissue disrupting apparatus 2. In other embodiments, tissue disrupting apparatus 2 may comprise a grip or structure located about outer tube 4 to facilitate handling by the user, while the proximal end of outer tube 4 is attached to a benchtop or cart-based machine, for example, or other type of mounted or fixed machinery. In these embodiments, the grip may or may not contain any other components of the tissue disrupting apparatus, such as a motor, while the machinery at the proximal end of outer tube 4 may contain one or more other components, for example, such as a suction system or various radiofrequency ablation components. In some embodiments, housing 6 may have a length of about 1 cm to about 12 cm, sometimes about 2 cm to about 8 cm, and other times about 3 cm to about 5 cm. The average diameter of the housing (or other transverse dimension to the longitudinal axis of the housing) may be about 1 cm to about 6 cm or more, sometimes about 2 cm to about 3 cm, and other times about 1.5 cm to about 2.5 cm. Housing 6 may further comprise one or more ridges, recesses or sections of textured or frictional surfaces, including but not limited to styrenic block copolymers or other polymer surfaces.

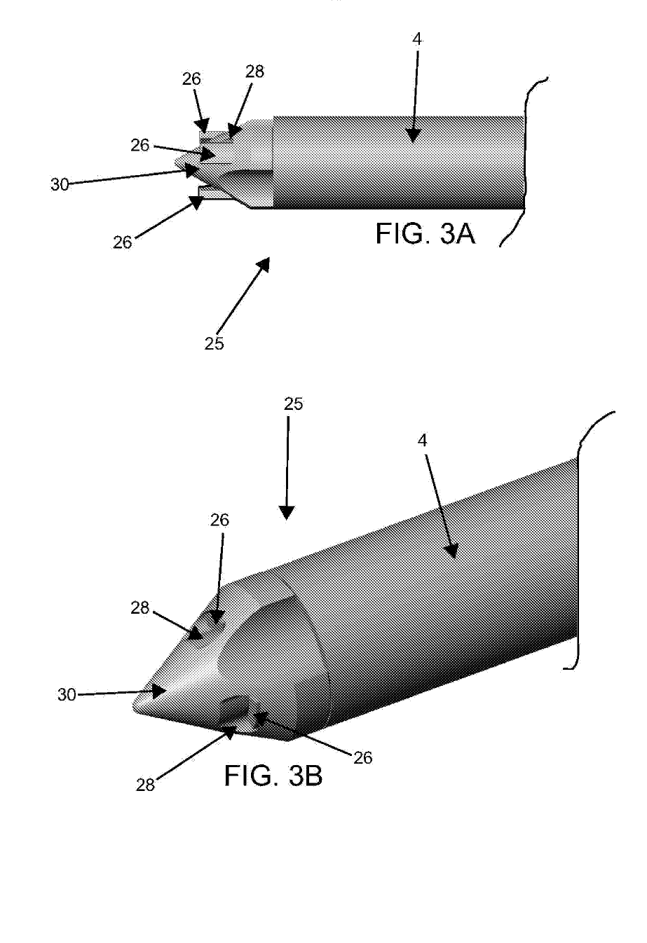

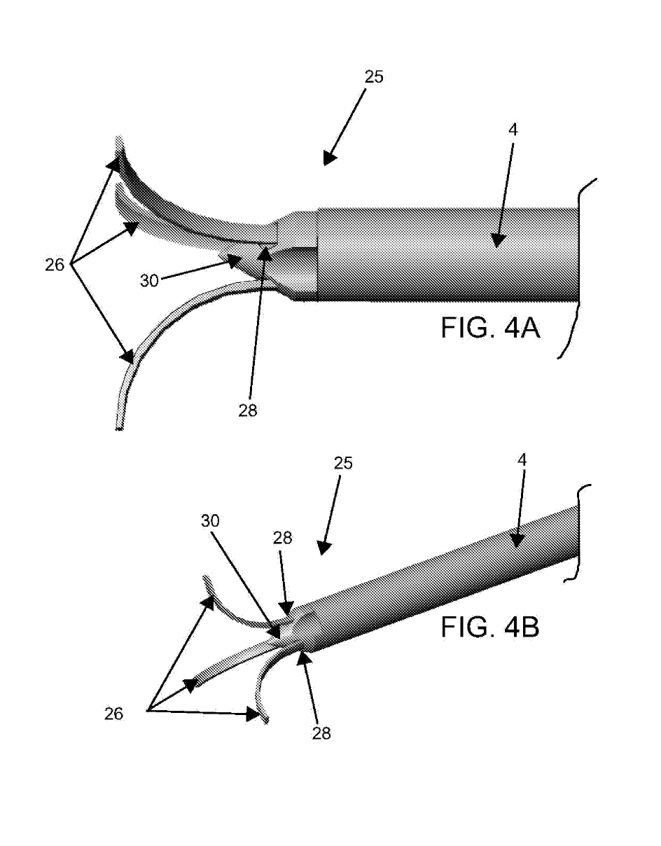

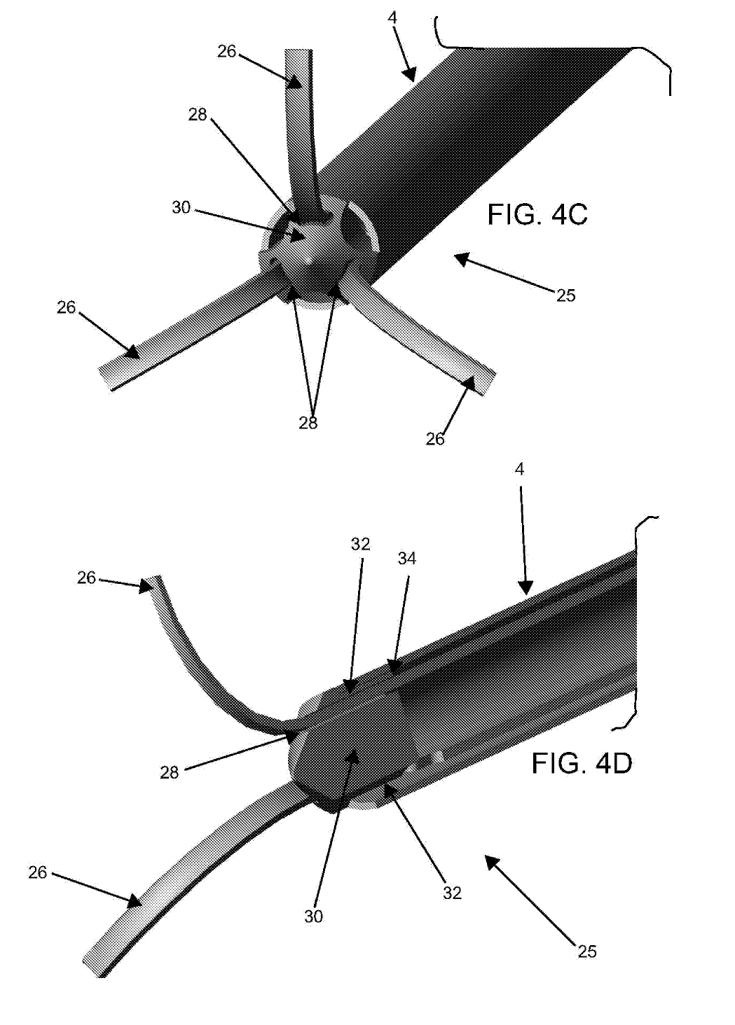

FIGS. 3A to 4D depict one embodiment of a tissue disrupting element 25, comprising one or more extension members 26 which may be retracted or extended from one or more extension apertures 28. In the retracted position, tissue disrupting element 25 may be inserted into and withdrawn from the body or cannula while reducing any mechanical interference from extension members 26. When extended and rotated or otherwise moved, extension members 26 may be used disrupt body tissues or structures by the repeated impact of the extension members 26 while rotated.

In FIGS. 3A to 4D, tissue disrupting element 25 comprises a head 30 with a conical configuration and three extension apertures 28 positioned at similar longitudinal positions of head 30 and equally spacing around the central axis of conical head 30. In other embodiments, however, head 30 may have a different configuration, including but not limited to a dome configuration, a concave configuration, a cube configuration, etc. In other embodiments, head 30 may comprise multiple points or edges that may be used to cut, chop, grind, emulsify or otherwise disrupt tissue or body structures separate from extension members 26. In still other embodiments, head 30 may comprises surfaces with a grit that may be used as a burr mechanism. The grit number may range from about 60 to about 1200, sometimes about 100 to about 600, and other times about 200 to about 500.

Head 30 may be optionally configured for tissue disrupting or disruption when extension members 26 are in their retracted configuration. For example, head 30 may be provided with cutting edges or grinding surfaces that may be used when tissue disrupting apparatus 2 is actuated. In some embodiments, the cutting edges or grinding surfaces may be used with extension members 26 in their extended configuration as well.

Head 30 may optionally comprise a port or aperture which may be used to perform suction or aspiration at the target site and/or to perfuse saline or other biocompatible fluids or materials to the target site. Use of saline or other cooling materials, for example, may be used to limit any thermal effect that may occur from frictional or other forces applied to the target site during removal procedures. The saline or other materials may or may not be chilled. In other embodiments, one or more therapeutic agents may be provided in the saline or fluid for any of a variety of therapeutic effects. These effects may include anti-inflammatory effects, anti-infective effects, anti-neoplastic effects, anti-proliferative effects, hemostatic effects, etc. Head 30 may have an average diameter or average transverse dimension with respect to its central longitudinal axis, of about 0.02 cm to about 2 cm, sometimes about 0.3 cm to about 1.5 cm, and other times about 0.04 cm to about 1 cm.

As shown in FIGS. 3A to 4D, extension members 26 may be configured to extend in a radially outward when in their extended position. The degree of curvature or deflection may range from about −150 degrees to about +150 degrees from the central axis of head 30 or outer tube 4, sometimes about −90 degrees to about +90 degrees, and other times about 0 degrees to about +90 degrees. The number of extension members 26 may range from about one extension member to about fifty extension members or more, other times about two extension members to about eight extension members, and sometimes about 3 extension members to about six extension members. As shown in FIGS. 3A to 4D, extension members 26 may each comprise similar configured strip members, but in other examples, embodiments with two or more extension members may have heterogeneous configurations, lengths, cross-sectional areas and shapes. The configuration or dimensions of an extension member need not be uniform along the longitudinal length of the extension member. In some embodiments whose extension members comprise strip members, the width of the strips may be about 1.5 times to about 10 times greater than the thickness of the strips. In other embodiments, the width of the strips may be about 2 times to about 6 times greater than the thickness, and other times about 3 times to about 5 times greater width than thickness. In other embodiments, extension members 26 may comprise wire members, tube members or blade members. Extension members 26 may comprise any of a variety of materials, including but not limited to nickel-titanium alloys, stainless steel, cobalt-chromium, polymers such as vinyl or nylon, or combinations thereof. The flexibility or rigidity of extension members 26 may vary, depending on the intended tissue or body material to be removed. In some embodiments, one or more extension members 26 may have a thickness of about 0.05 cm to about 0.5 cm or more, sometimes about 0.1 cm to about 0.3 cm, and other times about 0.15 cm to about 0.2 cm. In the fully extended configuration, extension members 26 may have a longitudinal length component (as measured along the longitudinal axis of outer tube 4) of about 0.01 cm to about 2 cm, sometimes about 0.1 cm to about 1 cm, and other times about 0.2 cm to about 0.5 cm. In the fully extended configuration, extension members 26 may have an overall radius (as measured from the central longitudinal or rotational axis of head 30 or outer tube 40 of about 0 cm to about 2 cm, sometimes about 0.1 cm to about 1 cm, and other times about 0.2 cm to about 0.5 cm. Although extension members 26 have a generally planar configuration in FIGS. 3A to 4D, in other embodiments, extension members 26 may curve or angle out of plane, particularly in the extended configuration.

In some embodiments, extension members 26 may be independently retracted and extended. In other embodiments, extension members 26 may be retracted and extended as groups. For example, a base member that is axially movable within outer tube 4 may be coupled to two or more extension members 26 to facilitate changes in the configuration of extension members 26.

Referring to FIG. 4D, extension apertures 28 are outer openings of extension lumens 32. As depicted in FIG. 4D, in this embodiment, extension lumens 32 are generally straight and have a parallel orientation with respect to the longitudinal axis of outer tube 4 or the rotational axis of head 30. In other embodiments, extension lumens 32 may be non-linear or curved. In still other embodiments, extension lumens 32 may be angled toward or away from the rotational axis of head 30, and/or may be angled in a clockwise or counter-clockwise direction, as determined from the proximal end 34 of extension lumen 32 to extension aperture 28.

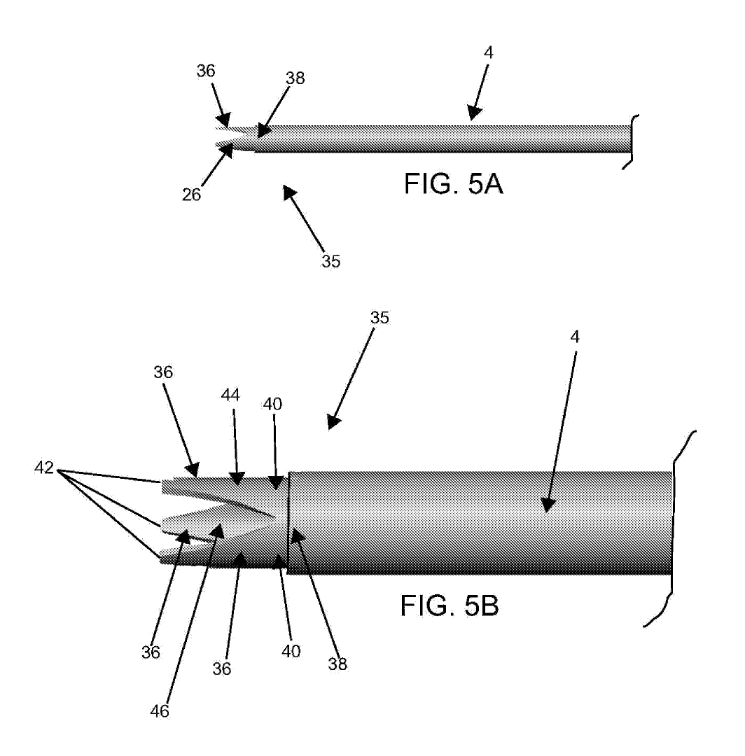

FIGS. 5A and 5B depict another example of a tissue disrupting element 35, comprising one or more tooth or protruding members 36. Protruding members 36 may be fixed in that they do no extend or retract. Protruding members 36 may also be rigid or deformable. In some embodiments, for example, protruding members 36 may deflect radially inward or outward upon impact against certain body structures such as bone. In this example, three protruding members 36 are provided on the distal section 38 of outer tube 4, but in other embodiments, the number of protruding members 36 ranges from about two to about ten or more, sometimes about three to about six, and other times about three to about five. In other embodiments, protruding members 36 may, instead of rotating, reciprocate back and forth or vibrate at a rate of about 1 Hz to about 100 MHz, sometimes about 60 Hz to about 6 MHz, and other times about 60 kHz to about 600 kHz. The amplitude or magnitude of displacement may be about 0.01 mm to about 10 mm, sometimes about 0.05 mm to about 5 mm, and other times about 0.1 mm to about 1 mm.

Protruding members 36 may have uniform or non-uniform cross-sectional dimensions along their longitudinal lengths. In the specific embodiment depicted in FIGS. 5A and 5B, protruding members 36 taper from their base 40 to their distal end 42. Distal ends 42 may be blunt or sharpened. Edges 44, 46 of protruding members 36 may also be blunt or sharpened. In embodiments where the tissue disrupting apparatus is configured to rotate in both directions, edges 44 and 46 may be configured differently to provide different cutting, chopping or grinding characteristics, depending on the rotation direction. Also, in FIGS. 5A and 5B, protruding members 36 have an outer surface 44 that is generally parallel to the longitudinal axis of outer tube 4, but in other embodiments, outer surface 44 may be angled radially inward or outward. Similarly, inner surface 46 of protruding members 36 may be angled inward or outward, or may be parallel to the longitudinal axis of outer tube 4. Also, protruding members 36 may have a generally straight configuration, as depicted in FIGS. 5A and 5B, or may have an angled or twisted configuration. The configuration and dimensions of each protruding member 36 need not be the same. In some embodiments, protruding members 36 may have an average length of 0.2 cm to about 2.5 cm or more, sometimes about 0.5 cm to about 2.0 cm, and other times about 1 cm to about 1.5 cm.

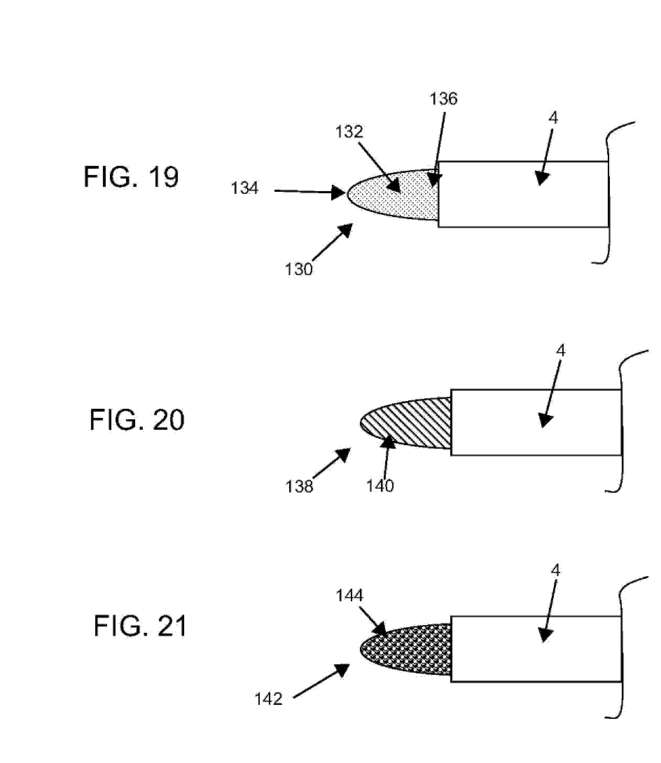

FIGS. 19 and 20 depict other embodiments of a tissue disrupting apparatus, comprising non-deformable or non-expandable tissue disrupting elements. FIG. 19, for example, depicts an embodiment comprising a tapered and rounded head 130 with a textured or grit surface 132. Surface 132 in FIG. 19 comprises a uniform grit type and density, but in other embodiments, the grit characteristics at the distal end 134 of head 130 may be different than the grit characteristics at the base 136 of head 130, for example. FIG. 20 depicts another embodiment of a tissue disrupting apparatus, comprising a fixed configuration head 138 with one or more cutting, chopping or debriding edges 140. The sharpness, angle or other configuration of edges 140 may vary along its length or the region of head 138. FIG. 21 depicts an embodiment comprising a head 142 with a plurality of grinding members 144. Grinding members 144 may have a homogeneous or a heterogeneous configuration and/or spacing. The degree of sharpness or cross-sectional shape of heads 130, 138 and 142 may vary in other embodiments.

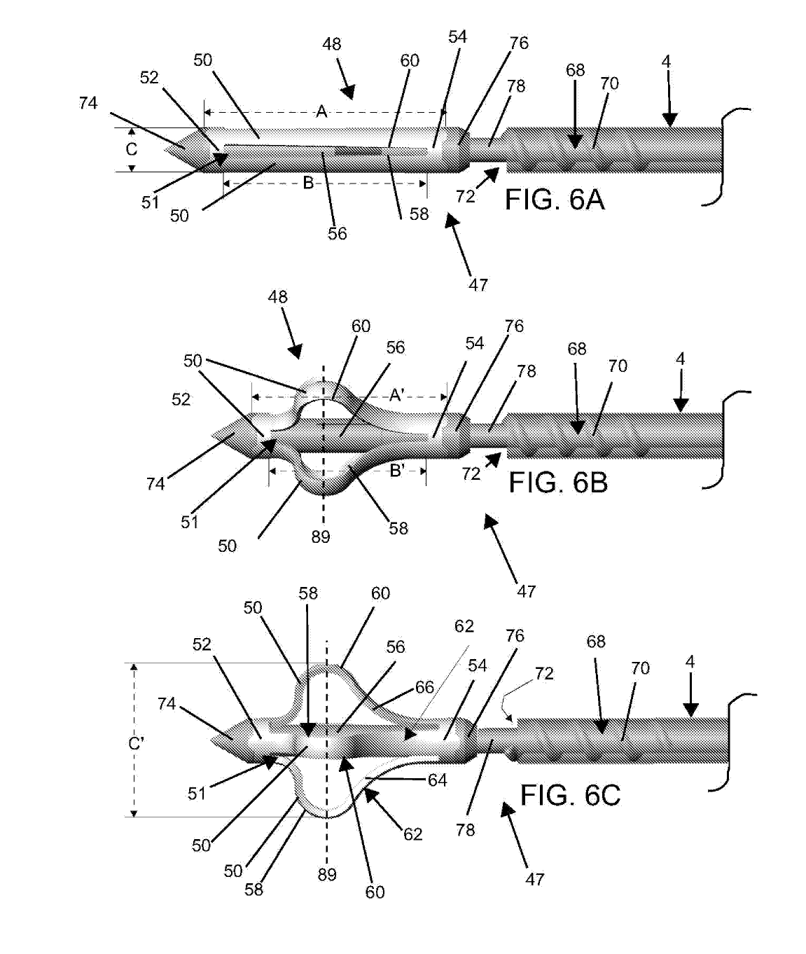

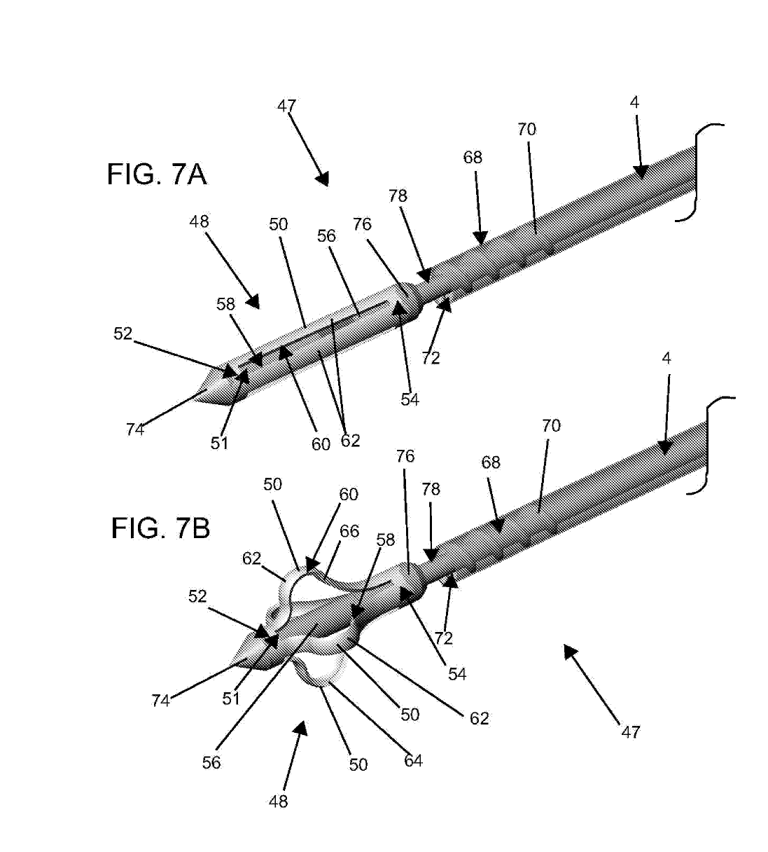

Referring to FIGS. 6A to 6C, in another embodiment, the tissue disrupting element 47 may comprise an expandable cage 48 with one or more longitudinal or elongate disrupting members 50 that have a reduced configuration, as shown in FIG. 6A, and an expanded configuration, as shown in FIGS. 6B and 6C. FIGS. 7A and 7B are perspective views of tissue disrupting element 47 depicted in FIGS. 6A to 6C. In the particular embodiment depicted in FIGS. 6A to 6C, the degree of expansion may be controlled by changing the distance between the distal end 52 and proximal end 54 of expandable cage 48. This distance may be changed with an adjustment member 56 that is axially coupled to distal end 52 of expandable cage 48. Adjustment member 56 may comprise a pull wire or tension element, with an optional overtube to protect the pull wire. The axial coupling between adjustment member 56 and distal end 52 permits rotation of expandable cage 52 without substantially altering the desired setting of adjustment member 56. In other embodiments, a portion or all of adjustment member 56 may rotate with expandable member 48 and an axial coupling may be provided within or proximate to the adjustment member 48, e.g. at the coupling between adjustment member 48 and its control interface located along outer tube 4 or in housing 6, for example.

Adjustment member 56 may be configured to limit the range of expansion of expandable cage 48. In other embodiments, one or more proximal controls of expandable cage 48 may limit its expansion range. In some embodiments, limiting the range of expansion may limit the stress acting on disrupting members 50, which may reduce the risk of fracture or failure during use. In some embodiments, expandable cage 48 in a reduced configuration may have a longitudinal dimension A of about 4 mm to about 20 mm or more, sometimes about 5 mm to about 15 mm, and other times about 6 mm to about 10 mm, and in an expanded configuration may have a longitudinal dimension A′ of about 3 mm to about 16 mm, sometimes about 4 mm to about 12 mm, and other times about 5 mm to about 8 mm. In the reduced configuration, the longitudinal dimension B of the slots 51 between disrupting members 50 may have a length of about 3 mm to about 18 mm or more, sometimes about 4 mm to about 12 mm, and other times about 5 mm to about 8 mm. In the expanded configuration, the longitudinal dimension B′ of slots 51 about 2 mm to about 15 mm or more, sometimes about 3 mm to about 9 mm, and other times about 4 mm to about 6 mm. In some embodiments, in the reduced configuration, expandable cage 48 has an average outer diameter C or transverse dimension with respect to the longitudinal dimension of about 0.75 mm to about 4 mm, sometimes about 1 mm to about 3 mm, and other times about 1.2 mm to about 1.5 mm, while in the expanded configuration may have an average outer diameter C′ or transverse dimension with respect to the longitudinal dimension of about 1.2 mm to about 10 mm, sometimes about 2 mm to about 8 mm, and other times about 4 mm to about 6 mm. As shown in FIGS. 6A to 7B, slots 51 between disrupting members 50 have a generally longitudinal configuration, but in other embodiments, slots 51 may be curved, helical or angled in reduced and/or expanded configuration.

In some embodiments, the percentage change of the longitudinal dimension of expandable cage 48 from its reduced configuration to its expanded configuration is about 10% to about 40%, sometimes about 12% to about 25%, and other times about 15% to about 20%, while the percentage change of disrupting member 50 from its reduced configuration to its expanded configuration is about 12% to about 50%, sometimes about 15% to about 30% and other times about 20% to about 25%. In some embodiments, the percentage change of the outer diameter or transverse dimension to the longitudinal dimension of expandable cage 48 or disrupting member 50 from its reduced configuration to its expanded configuration is about 25% to about 400%, sometimes about 100% to about 300%, and other times about 200% to about 250%.

Although expandable cage 48 has a reduced configuration and an expanded configuration, expandable cage 48 may have a native or natural configuration in which the stress acting on expandable cage 48 or disrupting members 50 is reduced compared to other configurations. In some embodiments, the native configuration comprises a configuration between the reduced configuration and the expanded configuration. With this particular embodiment, stress is exerted on expandable cage 48 in both the reduced configuration and the expanded configuration. In other embodiments, however, the native configuration may be about the same as the either the reduced configuration or the expanded configuration. With these latter designs, expandable cage 48 may exhibit reduced or no stress while in one configuration, while exerting higher levels of stress in the opposite configuration. For example, when the native configuration is close or the same as the expanded configuration, little if any stress may be exerted on disrupting members 50 when in the expanded configuration, but larger amounts of stress are exerted on disrupting members 50 when expandable cage 48 is collapsed into its reduced configuration. In some embodiments, this particular native configuration may be beneficial during use because the low or zero baseline stress acting on disrupting members 50 in its expanded configuration provides greater stress tolerance from impacting tissues or bone without stressing disrupting members 50 beyond their fracture point. Although collapsing expandable cage 48 to the reduced configuration may result in a greater magnitude of stress acting on expandable cage 48, the stress may be a transient stress that only occurs during insertion and removal of tissue disrupting apparatus 2, and with limited or little other stresses acting on expandable cage 48 during insertion and removal.

To produce expandable cage 48 with a particular native configuration, the manufacturing steps may vary depending upon the particular material or composition used for expandable cage 48. In embodiments where expandable cage 48 comprises stainless steel or nickel-titanium alloys, for example, a series of deformation steps and heat annealing steps may be used to form expandable cage 48 in a native, expanded configuration from a slotted tube configuration.

In FIGS. 6A to 7B, expandable cage 48 comprises four slits 51 with four disrupting members 50, but in other embodiments the number of disrupting members 50 may be different. Some embodiments may comprise anywhere from about one disrupting member 50 to about 20 or more disrupting members 50, while other embodiments may comprise about two or three disrupting members to about eight disrupting members, or sometimes about 4 disrupting members to about 6 disrupting members.

The disrupting members of the expandable cage may have a uniform cross-sectional size and shape along a substantial portion of the longitudinal length of each disrupting member, but in other embodiments, the cross-sectional size and shape of the disrupting members may vary along their longitudinal lengths. In FIGS. 6A to 7B, for example, disrupting members 50 comprise a tapered configuration. The widths of disrupting members 50 decrease from proximal end 54 to distal end 52 of expandable cage 48, while slots 51 taper from distal end 52 to proximal end 54. Disrupting members 50 may have any of a variety of cross-sectional shapes, including but not limited to squared, rectangular, trapezoidal, circular, elliptical, polygonal, and triangular, for example. The cross-sectional shape or size may vary along the length of disrupting member 50. In some embodiments, disrupting members 50 may be micropolished. Micropolishing may or may not reduce the risk of chipping or fragment formation when used to debride harder or denser body structures or tissues. The configurations of disrupting members 50 need not be generally the same for each, and may vary in one or more parameters. In some embodiments, a cutting edge 58 and 60 may be provided between the outer surface 62 and one or more side surfaces 64 and 66 of disrupting member 50. In some embodiments, cutting edge 58 or 60 may have an edge angle of about 90 degrees to about 10 degrees, sometimes about 75 degrees, to about 15 degrees, and other times about 60 degrees to about 30 degrees, and still other times about 45 degrees to about 40 degrees. As noted earlier, cutting edge 58 on one side of disrupting member 50 may have a different configuration than the opposite cutting edge 60, which may permit changes in the cutting, chopping, debriding, or emulsifying characteristics of expandable cage 48, depending upon its direction of rotation. In other embodiments, cutting edges 58 and 60 generally have the same features, but users may switch from one edge to the other when the first edge has worn out. In still other embodiments, the rotation direction may be selected depending upon the relative location of the tissue to be removed and any critical anatomical structures, such that if cutting edge 58 or 60 catches on the tissue or structure, tissue disrupting element 8 will be rotated away from the critical anatomical structure(s), if any.

As illustrated in FIGS. 6A to 7B, the tissue disrupting apparatus may optionally comprise a tissue transport assembly 68 which may be used to facilitate transport or removal of tissue within or along outer tube 4. In the particular embodiment depicted, tissue transport assembly 68 comprises a helical member 70 mounted on the drive member 78 which, when rotated in a particular direction, will mechanically facilitate proximal movement of tissue or other materials within the channel or lumen 72 of outer tube 4 occupied by helical member 70, as well as rotate expandable cage 48. When rotated in the opposite direction, helical member 70 may expel or distally transport tissue, fluid or other materials or agents from outer tube 4 or supplied to an infusion port of housing 6.

In some embodiments, helical member 70 may have a longitudinal dimension of about 2 mm to about 10 cm or more, sometimes about 3 mm to about 6 cm, and other times about 4 mm to about 1 cm. In other embodiments, the longitudinal dimension of helical member 70 may be characterized as a percentage of the longitudinal dimension of outer tube 4, and may range from about 5% to about 100% of the longitudinal dimension of outer tube 4, sometimes about 10% to about 50% or more, and other times about 15% to about 25%, and still other times is about 5% to about 15%. Although helical member 70 depicted in FIGS. 6A to 7B will rotate with expandable cage 48 due to its mounting onto common structure, drive member 78. In other embodiments, however, a helical member 70 may be rotate separately from drive member 70. For example, helical member 70 may comprise a helical coil located along at least a proximal portion of lumen 72 of outer tube 4 but is not mounted on drive member 70. In this particular example, helical member 70 can rotate independently of drive member 78. In still other embodiments, helical member 70 may be mounted on the surface of lumen 72 and can be used to transport tissue or substances along lumen 72 by rotation of outer tube 4, independent of drive member 78 or expandable cage 48.

Although helical member 70 is depicted as a continuous structure, in some embodiments, helical member 70 may be interrupted at one or more locations. Also, the degree or angle of tightness of helical member 70 may vary, from about 0.5 turns/mm to about 2 turns/mm, sometimes about 0.75 turns/mm to about 1.5 turns/mm, and other times about 1 turn/mm to about 1.3 turns/mm. The cross-sectional shape of helical member 70 may be generally rounded as depicted in FIGS. 6A to 7B, but in other embodiments, may have one or more edges. The general cross-sectional shape of helical member 70 may be circular, elliptical, triangular, trapezoidal, squared, rectangular or any other shape. The turn tightness and cross-sectional shape or area of helical member 70 may be uniform or may vary along its length. In some embodiments, multiple helical members 70 may be provided in parallel or serially within outer tube 4.

In some embodiments, drive member 78 is configured to extend distally and retract from outer tube 4 by a length of about 0.01 cm to about 2 cm or more, sometimes about 0.02 cm to about 1.5 cm and other times about 0.05 to about 1 cm. In some embodiments, helical member 70 is located proximal to the tissue disrupting element 8 at a distance of about 0.01 cm to about 2 cm or more, sometimes about 0.02 cm to about 1.5 cm and other times about 0.05 to about 1 cm. In some embodiments, when drive member 78 is maximally extended from outer tube 4, helical member 70 may protrude from outer tube 4 by a longitudinal dimension of about 0.01 cm to about 2 cm or more, sometimes about 0.1 cm to about 1 cm, and other times about 0.25 cm to about 0.5 cm. In some embodiments, the degree of extension of drive member 78 and/or helical member 70 may affect the degree of tissue transport by the tissue transport assembly.

The distal cap 74 and proximal cap 76 of tissue disrupting element 47 may be separately formed from expandable cage 48, but in other embodiments, distal cap 74 and/or proximal cap 76 may be integrally formed with expandable cage 48. In other embodiments, one or more disrupting members 50 may be individually formed and attached, for example, to distal and proximal caps 74, 76. The relationships between distal cap 74, proximal cap 76, and adjustment member 56 may vary, depending upon the particular embodiment. In one embodiment, for example, adjustment member 56 is coupled to drive member 78 at a mid portion of adjustment member 56, such that when adjustment member 56 is shortened, distal cap 74 is retracted relative to drive member 78 and proximal cap 76 is extended distally relative to drive member 70. In another embodiment, when adjustment member 56 is shortened, the relative location of distal cap 74 remains fixed while proximal cap 76 is extended distally relative to drive member 70, while in another embodiment, when adjustment member 56 is shortened, the relative location of proximal cap 74 remains fixed while distal cap 76 is retracted relative to drive member 78. The particular expansion profile may depend upon user preference and/or the visibility of the target tissue or adjacent structures such as nerves and blood vessels.

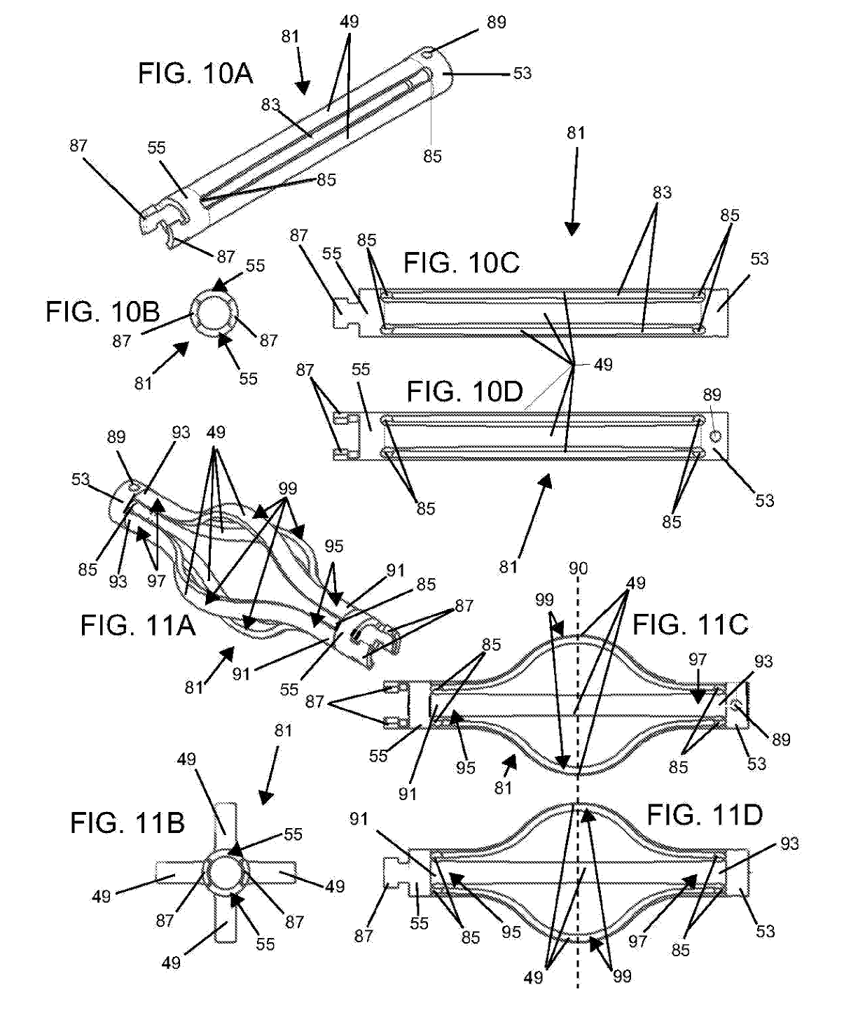

Referring now to FIGS. 10A to 10D and 11A to 11D, one embodiment of expandable cage 81 is depicted in its reduced and expanded configurations, respectively. In this embodiment, expandable cage 81 may be manufactured from a tubular body that is stamped, machine-cut or laser-cut with slits or slots 83 to form disrupting members 49 therebetween. In other embodiments, expandable cage 81 may be stamped, machine-cut or laser-cut from a sheet material where one edge is then welded or bonded to another edge to form a tubular structure. In some embodiments, slots 83 may be provided with rounded ends 85, as depicted in FIGS. 10A to 10D and 11A to 11D, but in other embodiments, slots 83 may have squared or tapered ends, for example. In some embodiments, rounded ends 85 may reduce stress acting on disrupting members 49 when collapsing and/or expanding disrupting members 49. Rounded ends 85 are depicted with a larger diameter than the width of slots 83, but in other embodiments, rounded ends 85 may have a diameter similar to the width of slots 83. Expandable cage 81 may be formed with tabs 87 and/or holes 89 to facilitate coupling of expandable cage 81 to adjustment member 57, distal cap 75 and/or proximal cap 77, or other components of the tissue disrupting apparatus.

In embodiments where the tubular body comprises a nickel-titanium alloy and has a native configuration that is substantially similar to the deployed configuration of expandable cage 81, the tubular body is deformed or shaped toward the deployed configuration by generating a strain in the tubular body by no more than about 8%. The tubular body is then heat annealed and cooled while strained to reduce the strain. The shaping and heat annealing procedure is repeated until the native configuration of expandable cage 81 is achieved.

In the embodiment of expandable cage 81, distal end 53 and proximal end 55 of expandable cage 81 are spaced about 1 mm from ends 85 of slots 83, but in other embodiments, ends 84 may be located anywhere from about 0.5 mm to about 10 mm or more from distal end 53 and proximal end 55, sometimes about 1 mm to about 3 mm, and other times about 1 mm to about 2 mm. The spacing at distal end 53 and proximal end 55 need not be the same. Although ends 53 and 55 in FIGS. 10A to 11D are integrally formed with disrupting members 49, in other embodiments, disrupting members 49 may be separately formed but attached to ends 53 and 55, or directly attached to caps 75 and 77.

As illustrated in FIGS. 11A to 11D, in some embodiments, disrupting members 49 in their expanded configuration have a generally bell-shaped curve, but in other embodiments may have a mushroom shape or an angular shape, for example. In the particular embodiment, the maximum transverse dimension 90 of expandable cage 81 is configured to be approximately 50% longitudinal dimension position of disrupting members 49. In other embodiments, however, from proximal to distal, the maximum transverse dimension 90 of expandable cage 49 may be located anywhere from about 0% to about 100% of the longitudinal dimension of disrupting members, and other times about 20% to about 80%, and still other times about 25% to about 50%. In FIGS. 6B and 6C, for example, expandable cage 50 is configured with a maximum transverse dimension 89 located about two-thirds of the distance from proximal end 54 to distal end 52 of expandable cage 48. In still other embodiments, a portion of one or more disrupting members may be located proximal to the proximal end of the expandable cage, and/or distal to the distal end of the expandable cage, by at least partially folding back upon itself in the expanded configuration. Embodiments where a portion of a disrupting member is distal to the distal ends of the expandable cage may be beneficial when disrupting shallow cavities or forming cavities in a tissue surface, with less interference from its proximal cap. In still other embodiments, disrupting members may have a generally zig-zag or generally sinusoidal profile in their expanded configurations.

Referring still to FIGS. 11C and 11D, the profile of disrupting member 49 of expandable cage 81 may be configured so that the proximal and the distal ends 91, 93 of disrupting members 49 may comprise end sections 95, 97 in addition to the bend zone 99. End sections 95, 97 may be generally linear or curved. In other embodiments, bend zone 99 may comprise a substantial length of disrupting member 49, or one or more of distal ends 91, 93 is omitted. In some further embodiments, end sections 95, 97 have less curvature than bend zone 99 of disrupting members 50. In some specific embodiments, sections 95, 97 are generally linear and generally oriented at a zero degree angle with respect to the distal and proximal ends 53, 55 of expandable cage 81, but in other embodiments, the angle between linear sections 95, 97 and ends 53, 55 may range from about 0 degrees to about 120 degrees, sometimes about 0 degrees to about 20 degrees, and other times about 5 degrees to about 25 degrees. The angle between distal end 53 and linear section 95 need not be the same as the angle between proximal end 55 and linear section 97. Also, the length of linear sections 95 and 97 need not be the same in any one disrupting member 49 or between disrupting members 49. In some embodiments, the length of end sections 95, 97 are about 0.75 mm to about 5 mm, sometimes about 1 mm to about 3 mm, and other times about 1 mm to about 2 mm. In some embodiments, it is hypothesized that configuring expandable cage 81 with one or more straight portions may reduce the risk of breaking or fracturing disrupting members 49 by shifting bend zone 99 of disrupting members 49 away from the ends 53, 55 of expandable cage 81. In some instances, the curvature of bend zone 99 in the expanded configuration may introduce high stress concentrations. By configuring disrupting member 49 with end sections 95, 97 that displace bend zone 99 away from ends 53, 55 of expandable cage 81, the amount of stress acting at distal ends 91, 93 of disrupting member 49 may be reduced, which in turn may reduce the risk of fracture from the side stresses generated at ends 53, 55 of expandable cage 81 when expandable cage 81 is rotated at high speeds and impacted against bone or other tissue.

In the particular embodiment depicted in FIGS. 10A to 11D, expandable cage 81 comprises disrupting members 49 that bow or curve outward from the proximal and distal ends 55, 53 of expandable cage 81. When rotated, disrupting members 49 may define an internal volume of expandable cage 81. In some embodiments, this internal volume may be used to retain tissue or other material to be retrieved or removed from the target site or the body. In some embodiments, this internal volume may be about 0.001 cm³ to about 1.5 cm³ or more, sometimes about 0.01 cm³ to about 0.5 cm³, and other times about 0.02 cm³ to about 0.1 cm³, and still other times about 0.01 cm³ to about 0.05 cm³, where there is an interior space maintained for gathering the debris as tissue is removed. The size and shape of the internal volume may vary depending upon the lengths and orientations of end sections 95, 97 and bend zone 99, and the shape profile and expansion characteristics of disrupting members 49.

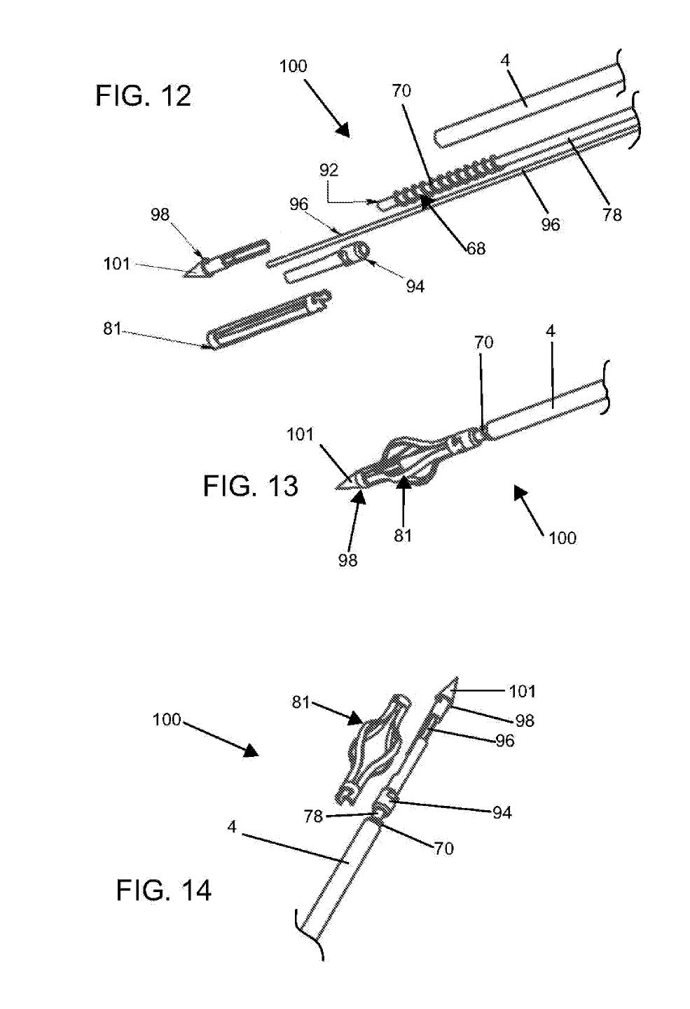

FIG. 12 depicts the components of one embodiment of a tissue disrupting apparatus 100 comprising expandable cage 81 of FIGS. 10A to 11D. In this particular embodiment, the distal end 92 of drive member 78 is attached to a proximal cage housing 94. A wire adjustment member 96 is attached to a distal cage housing 98, which further comprises a nose piece 101. In this particular embodiment, nose piece 101 has an optional pointed configuration, and may also be optionally configured with complementary recesses (not shown) for coupling tabs 87 of expandable cage 81 to nose piece 101. In some embodiments, a pointed or piercing configuration may be beneficial for stabilizing the tissue disrupting apparatus against a body structure during the tissue disrupting procedure, and/or to pierce the tissue or body structure to be removed. Proximal cage housing 94 may also have complementary mechanical interfit with the holes of expandable cage 81, if any. One of skill in the art will understand that in other embodiments, any of a variety of mechanical and frictional interfits and welding, soldering or bonding methods may be provided or used to attach these components. In this particular embodiment, drive member 78 and wire adjustment member 96 may be positioned within lumen 72 of outer tube 4. Outer tube 4 may be attached to housing 6. FIG. 14 depicts the components in FIG. 12 in an assembled state except for expandable cage 48, while FIG. 13 depicts all of the components in FIG. 12 in an assembled state.

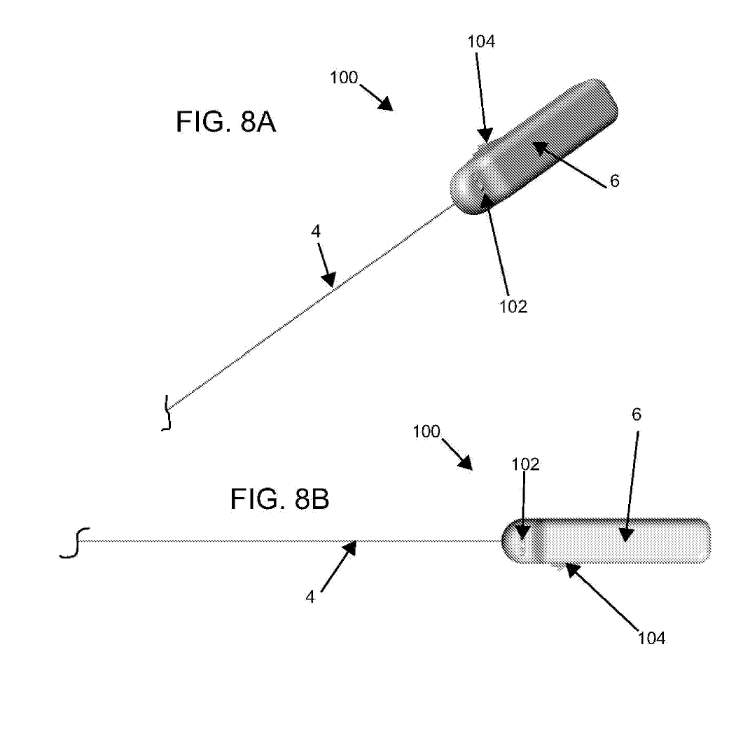

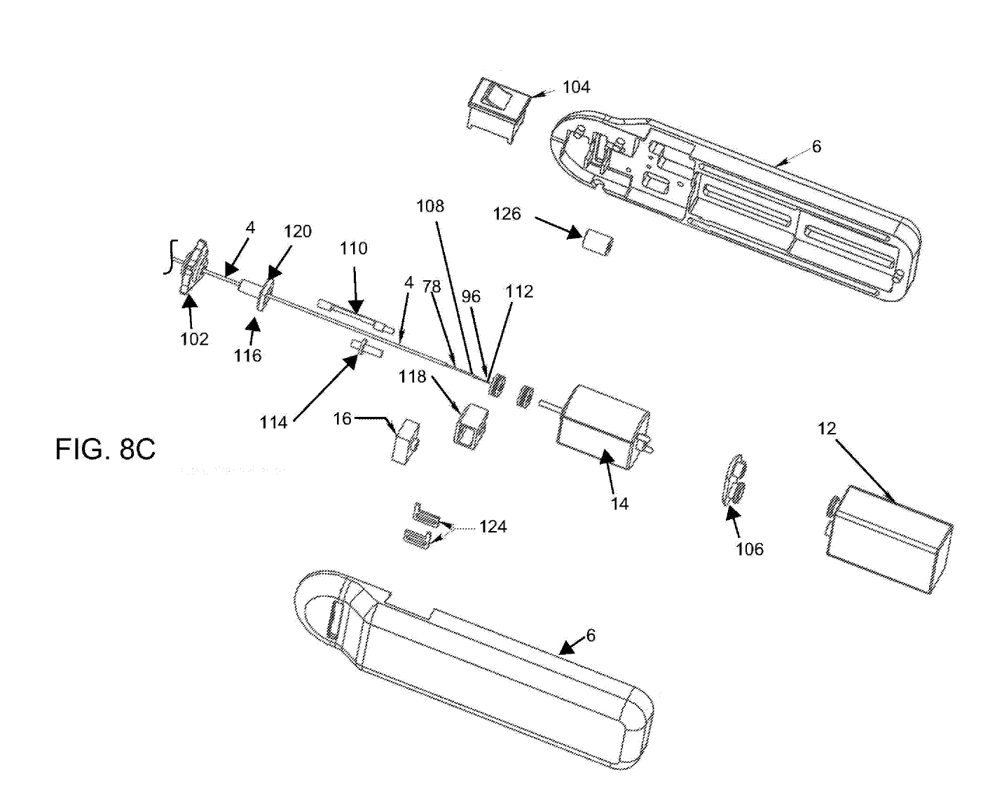

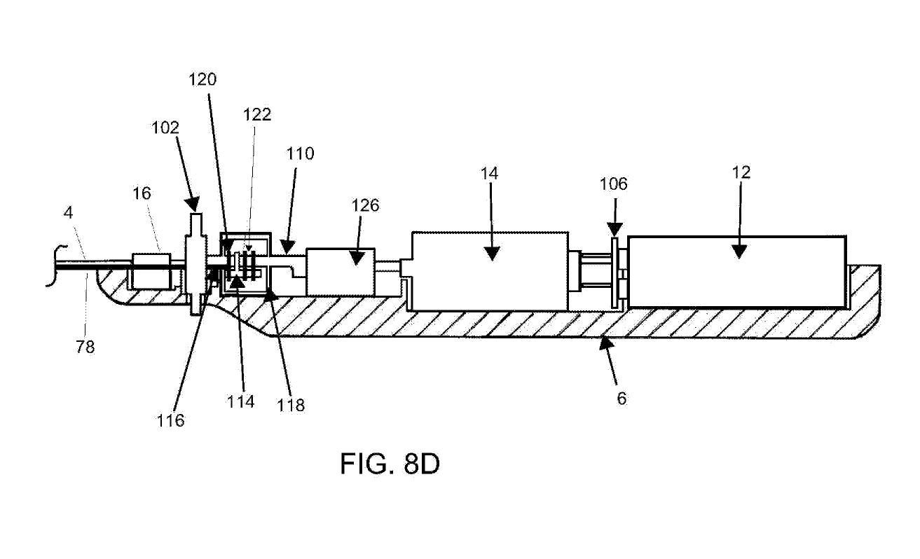

Referring to FIGS. 8A and 8B, in the embodiment of tissue disrupting apparatus 100, housing 6 comprises an adjustment mechanism with a thumbwheel 102 configured to adjust the collapse and expansion of expandable cage 81. Thumbwheel 102 may provide a continuous range of change to expandable cage 81, but in other embodiments, the turning of thumbwheel 102 may be configured with clicks or detents that provide one or more preset positions. As mentioned previously, in other embodiments, any of a variety of other control mechanisms and interfaces may be used. In some embodiments, the adjustment mechanism may comprise one or more blocking elements or other adjustment limiting configurations to resist or prevent overexpansion of expandable cage 81. In other embodiments described below, limit structures are provided in housing 6 to resist overexpansion of expandable cage 81. In this particular embodiment, tissue disrupting apparatus 100 is configured to rotate the tissue disrupting element at a fixed rotational speed. Rotation may be controlled using a rocker-type power switch 104. As mentioned previously, however, any of a variety of power and/or speed control mechanisms may be used.

Referring to FIGS. 8C and 8D, the components within housing 6 of tissue disrupting apparatus 100 from FIGS. 8A and 8B are described. FIG. 8C is a component view of the internal components of housing 6, while FIG. 8D is a schematic cross-sectional view with a portion of housing 6 removed. As shown in FIG. 8D, the proximal end 108 of drive member 78 is coupled to a driveshaft 110, while the proximal end 112 of adjustment member 96 extends from out of proximal end 108 of drive member 78 and is attached to a drive key 114. Thumbwheel 102 is movably coupled to a thrust member 116 so that the rotation of thumbwheel 102 results in axial movement of thrust member 116. In some embodiments, thrust member 116 may be configured with helical threads that are complementary to a threaded lumen of thumbwheel 102. In other embodiments, however, other structures for manipulating thrust member 116 may be used, including a slide or a pivot member. Thrust member 116 acts on drive key 114 by a retaining structure 118 which is configured to movably couple thrust member 116 to drive key 114. Retaining structure 118 permits the rotation of driveshaft 110 while also coupling the axial movements of thrust member 116 to drive key 114 to manipulate expandable cage 49. Thrust member 116 may comprise a flange 120 to facilitate retention of thrust member 116 with retaining structure 118. Flange 120 may comprise a bearing to facilitate any rotational movement of drive key 114 against flange 120. Retaining structure 118 may also contain one or more retaining bearings 122 to facilitate rotation of driveshaft 110 and drive key 114 while transmitting any axial forces to drive key 114. Retaining structure 118 is optionally provided with one or more limiters 124, which may be used to restrict overexpansion or collapse of expandable cage 49. Driveshaft 110 may be directly coupled to motor 14, or coupled using a coupler 126. Coupler 126 may be configured to permit some axial movement of driveshaft 110 in embodiments where driveshaft 110 is directly coupled to a control interface for manipulating expandable cage 81.

As illustrated in FIG. 8D, tissue disrupting apparatus 100 is powered using a battery 12 that is coupled to motor 14 using a battery connector 106. As depicted in FIG. 8C, battery 12 may be a standardized battery such as a 9-volt battery, or a customized battery. Other examples of drive shafts couplings and adjustment mechanisms that may be used are disclosed in U.S. Pat. No. 5,030,201, which is hereby incorporated by reference in its entirety.

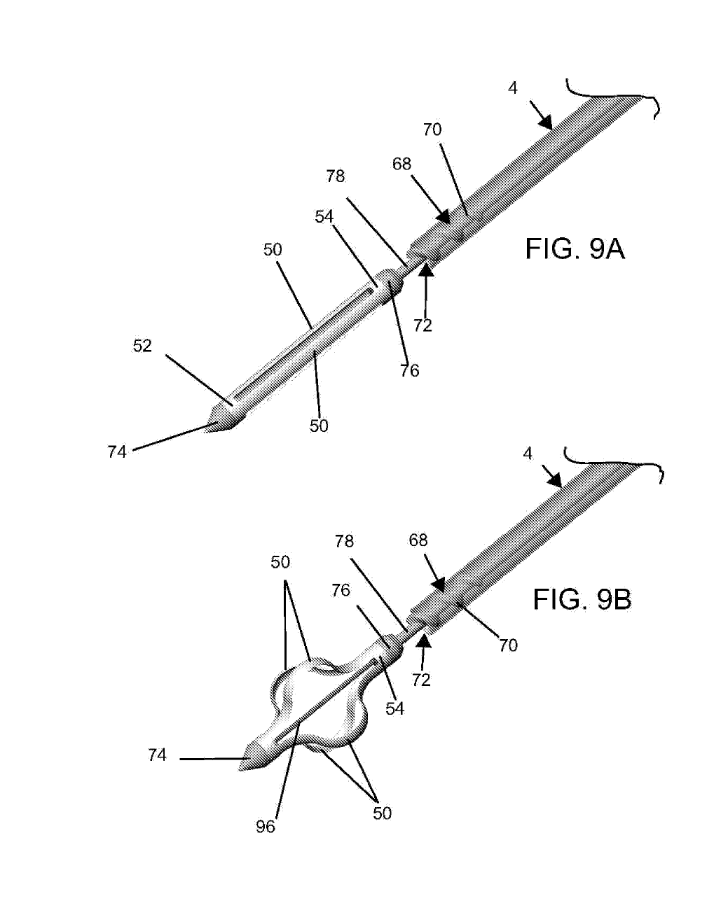

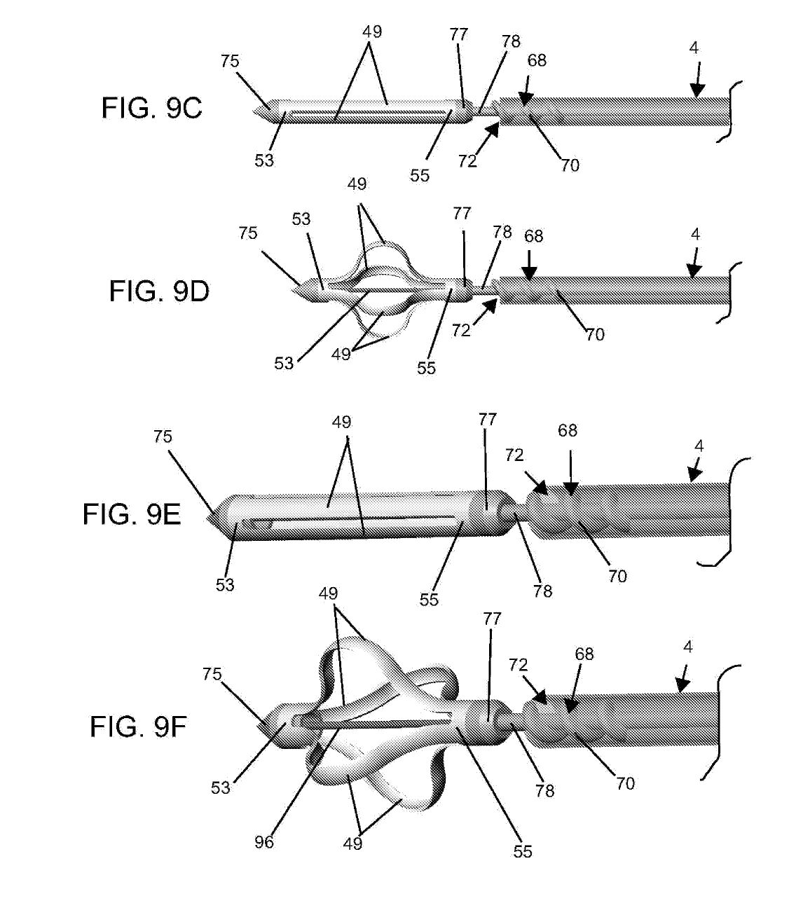

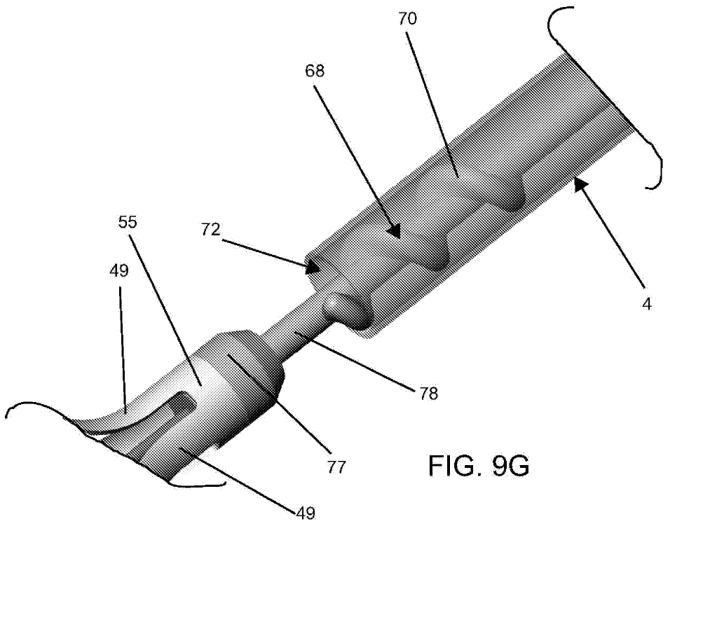

FIGS. 9A to 9F are various views of the distal components of tissue disrupting apparatus 100 in assembled form. As illustrated in these figures, outer tube 4 comprises lumen 72 which contains the drive member 78 coupled to expandable cage 81. Drive member 78 comprises tissue transport assembly 68, which comprises helical member 70. In this particular embodiment, helical member 70 is spaced apart from proximal cap 76 of tissue disrupting element 8 by about 0.8 mm, but in other embodiments, helical member 70 may be contacting tissue disrupting element 8 or spaced at a different distance from tissue disrupting element 8.

Expandable cage 81 in FIGS. 9A to 9F comprises four disrupting members 49 which taper proximal to distal from a width of about 0.7 mm to about 0.4 mm, but in other embodiments, may be non-tapering or may taper to a greater or lesser degree. The magnitude of variation may range from about 0% to about 50% or more, sometimes about 5% to about 40% and other times about 0% to about 25%. The tapering of disrupting members 49 in FIGS. 9A to 9F is also illustrated in FIG. 17, which depicts three-dimensional expandable cage 81 as a two-dimensional schematic template. As mentioned previously, expandable cage 81 may be formed from a tubular body 80 or from a sheet of material that is then rolled and joined along a pair of opposing edges. Slots 82 in tubular body 80 may form a 90 degree angle to the outer surface of the expandable cage 81, or may have an undercut or beveled cut. In some embodiments, where expandable cage 81 is formed from a sheet of material, a slight undercut may be provided to slots 82 such that when the sheet of material is formed into a tube, the sidewalls of slot 82 are normal to the outer surface of cage 81, or at some other desired angle.

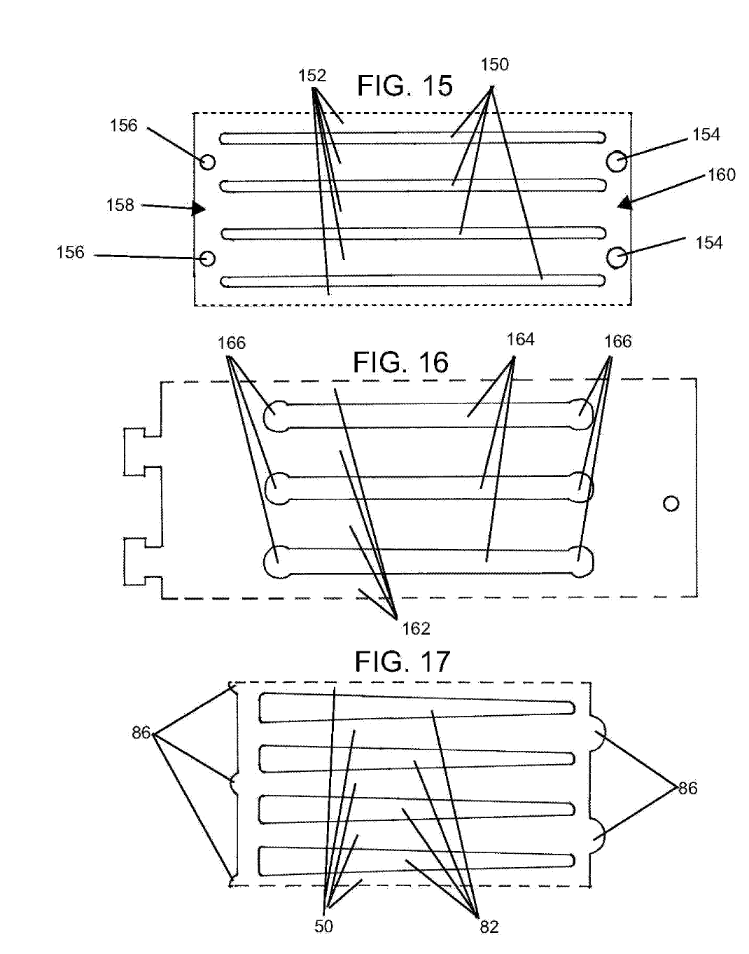

FIGS. 15 and 16 depict other embodiments of expandable cage 146, 148. In FIG. 15, for example, slots 150 and disrupting members 152 have a generally uniform width along a substantial portion of their longitudinal lengths. In this particular embodiment, the width of disrupting member 152 is about four times greater than the width of slots 150, but in other embodiments, the width ratio is about 0.8 times to about 5 times greater or more, sometimes about 1 time to about 4 times greater, and other times about 1.2 times to about 2 times greater. In some embodiments, one or more slots 150 or disrupting members 152 may have a different configuration than other slots 150 and disrupting members 152. The embodiment of expandable cage 146 in FIG. 15 comprises holes 154 and 156 for attaching expandable cage 146 to the rest of the tissue disrupting apparatus. Holes 154 and 156 may have a uniform or heterogeneous size or shape. The number of tabs, holes or other attachment structures of expandable cage 146 need not be equal in number between the proximal and distal ends 158 and 160 of expandable cage 48. FIG. 16 depicts another embodiment of expandable cage 148, comprising three disrupting members 162 and three slots 164. In this particular embodiment, slots 164 comprise rounded ends 166 with a larger diameter than the width of slots 164. Rounded ends 166 located at the proximal end 168 of expandable cage 148 need not have the same size or configuration as those located at the distal end 170 of expandable cage 148.

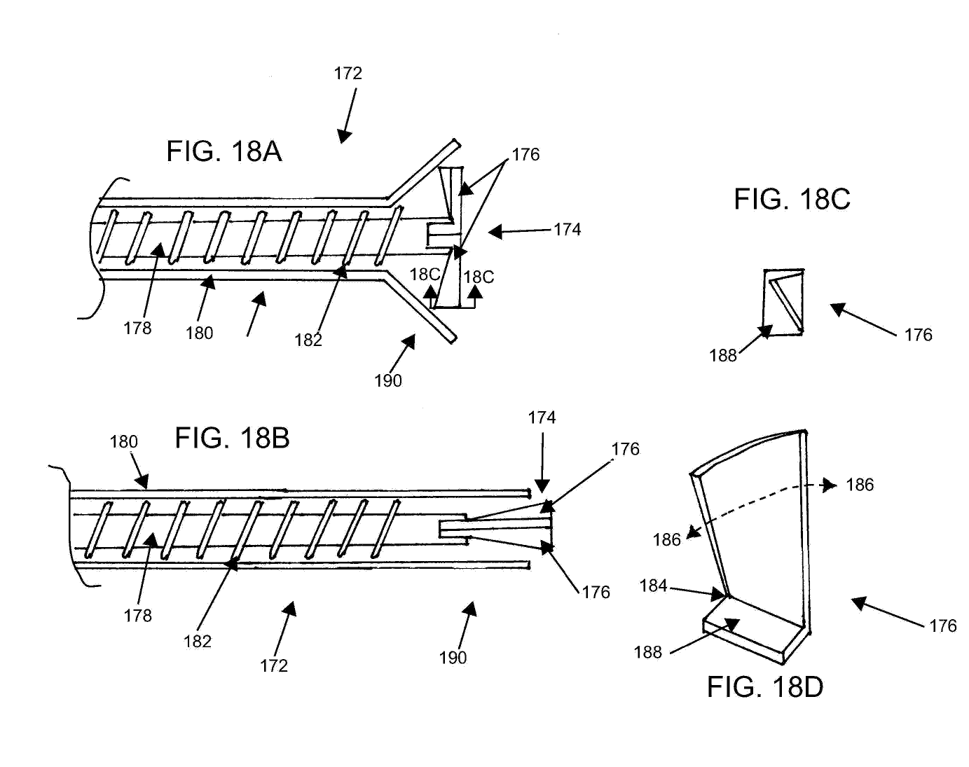

In another embodiment illustrated in FIGS. 18A to 18D, tissue disrupting apparatus 172 comprises a disrupting assembly 174 with one or more blades 176. In some embodiments, the number of blades 176 may range from about two to about eight, sometimes from about two to about four, and other times from about two to about 3. Blades 176 may be coupled to drive member 178 by mechanical interfit and/or bonding/welding. Drive member 178 resides in a tubular body 180 and optionally comprises a helical member 182. Blades 176 illustrated in FIGS. 18A to 18D have a generally rectangular configuration, but in other embodiments, the blades may be petal-like, rounded, or have any other configuration. Blades 176 may comprise one or more angles 184 or curves 186. In some embodiments, an angle or a curve may be provided to facilitate fluid or material movement toward helical member 182. A fold or curve may also be used to provide a mounting flange 188 or other type of protrusion configured to mount blade 176 into or into drive member 178. When rotated in the opposite direction, curve 186 of blade 176 may be beneficial, for example, for eluting fluid or therapeutic agents from tissue disrupting apparatus 172, or for dispersing or scattering connective tissue, floaters, blood clots and other materials away from tissue disrupting apparatus 172.

The length of blade 176 may vary depending upon the particular configuration and clinical indication. In FIGS. 18A and 18B, tissue disrupting apparatus 172 comprises deformable blades 176 that may be collapsed and expanded. Blades 176 may comprise any of a variety of suitable materials, including but not limited to nickel-titanium, stainless steel, cobalt-chromium, and nickel-cobalt-chromium-molybdenum. In some embodiments, blades 176 may be used with a fixed diameter outer tube to collapse blades 176 into their reduced configuration during insertion into the body. In other embodiments, blades 176 may be used with a tubular body 180 comprising an expandable funnel 190. In this embodiment, both funnel 190 and blades 176 are collapsed during insertion, but both expand when extended from the tissue disrupting apparatus 172. Embodiments with funnels 190 may be beneficial to protect adjacent body structures from inadvertent blade damage during use. Funnel 190 may comprise any of a variety of resilient materials. Various funnel-type designs that may be adapted for use in one or more embodiments are described in U.S. Pat. No. 7,108,705 and U.S. Pat. No. 5,460,170, which are hereby incorporated by reference in their entirety.

The tissue disrupting devices described herein may be used or adapted for use in a variety of medical procedures, which may be less invasive than traditional surgeries and speed recovery from such procedures.

For example, in one embodiment, a patient is prepped and draped in sterile fashion, and local, regional or general anesthesia is achieved. A guidewire or trocar is inserted at a desired target site and the location of the guidewire is confirmed. A cannula or introducer is passed over the guidewire and the guidewire is removed. A tissue disrupting apparatus comprising an expandable disrupter is collapsed into a delivery configuration and then inserted into the cannula or introducer. In other embodiments, the cannula, introducer, or tissue disrupting apparatus may be inserted directly to the target site without the prior insertion of other guidance components to the target site. After verifying the placement of the expandable disrupter, the expandable disrupter is expanded and the tissue or structure to be removed is contacted by the expanded disrupter. In some embodiments, the spatial relationship(s) between the disrupter and the other anatomical landmarks are identified, and the rotational direction of the disrupter is selected based upon the spatial relationship(s). In some embodiments where the tissue disrupting apparatus comprises a tapered or pointed head, or any other type of slip-resistant head, the head of the tissue disrupting apparatus may be pushed against a body tissue or structure before or during rotation of the disrupter to maintain the disrupter position. In some embodiments where the head of the tissue disrupting apparatus comprises a grit or other textured surface or structure, rotation of the head may be used to remove or alter body tissue before, during or after expansion of the expandable disrupter. Suction or mechanical aspiration may be applied as needed before, during or after a period of disrupting to remove any disrupted tissue or to clean or clarify the target site. In some instances, fluid or other matter may be infused before, during or after a period of disrupting to clean, clarify or treat the target site. The disrupter may be repositioned as needed to perform additional tissue disrupting. In some instances, the disrupter may continue rotation during repositioning, but in other embodiments, the rotation may be stopped during repositioning. In some embodiments, the disrupter may be collapsed prior to repositioning, but in other embodiments, may be repositioned in the expanded configuration.

In some embodiments, the tissue disrupting apparatus may be rotated as a higher speed to generate thermal or frictional energy. The energy may be used to modulate tissue response at the target site or used to achieve hemostasis at the target site.

Once tissue disrupting is completed, and adequate hemostasis of the target site is achieved, the disrupter is collapsed and withdrawn from the introducer or cannula. In some embodiments, one or more deep sutures or tissue anchors may be placed along the insertion pathway to facilitate wound closure. In other embodiments, one or more wound drains may be placed into the insertion pathway before, during or after removal of the introducer or cannula.

The above procedure may be used to treat or diagnosis any of a variety of conditions, including but not limited to dermatologic, central and peripheral neurological, gastrointestinal, traumatic, musculoskeletal, rheumatological, nephrological, neoplastic, inflammatory, auto-immune, vascular and other conditions. Methods for accessing the spine, for example, are also described in U.S. Pat. Pub. 2006/0206118, U.S. Pat. Pub. 2007/0213583 and U.S. Pat. Pub. 2007/0213584, all of which are hereby incorporated by reference in their entirety. Two examples of using a tissue disrupting apparatus are also discussed below.

### Spinal Procedures

Expandable tissue disrupting devices may also be useful in orthopedic procedures. For example, discectomy procedures can be invasive on various levels and use instruments of varying size. One disadvantage of using traditional surgical instruments and more open procedures is that they may cause greater alteration of the spinal anatomy to achieve access to the target site. Traditional surgeries and open procedure often require separating muscle and connective tissue from the spine in order to provide adequate exposure at the surgical site and to avoid damage to neurovascular structures. Also, traditional surgical instruments may create tissue fragments and remnants that need to be collected during the dissection and treatment processes. Two different instruments may be used and interchanged: one for excising the tissue, another for collecting the loose tissue. This can be complicated when working with several instruments at once or having to continuously switch between instruments.

In one embodiment, a disrupter may be used as a surgical tool to perform two functions in a discectomy procedure, disrupting tissue and gathering debris. The disrupter may be introduced into the disc through a cannula inserted into the site of surgery, but may also be used in an open surgery. The disrupter may be an expandable device that has a collapsed or “closed” state when inserted into the body or cannula, as well as an expanded or “open” state once positioned at the target site for cutting, chopping, grinding, burring, emulsifying or otherwise disrupting the disc material.

In one embodiment, methods of removing material from a spinal column of a human or an animal are provided. Such methods comprise placing into a spinal column, for example an intervertebral disc, an outer housing with a rotational element disposed distally about a shaft, and rotating the rotational element relative to the outer housing. In some embodiments, a rotating element with an adjustable tissue disrupting feature may be provided, which may assist in transporting material from the intervertebral disc toward the outer housing with or without aid of supplemental aspiration. The method may further comprise passing the material from the body through the cannula.

The positioning of a tissue disrupting apparatus may comprise percutaneously advancing a tissue disrupting tip of the apparatus to a target site of the spine and positioning the tissue disrupting tip of the device in proximity to the intended material to be removed. This material may be, for example, a surface of a herniated disc, or the nucleus pulposus of a disc. In some embodiments, the tissue disrupting tip is adjustable in size and the tip and housing may be positioned relative to each other so that the rotation of the tissue disrupting tip is effective in emulsifying material and drawing the material from the target site of a human or an animal into the outer housing. The material from the target site may be removed by applying optional suction or mechanical aspiration to the distal tip.

The methods may further comprise applying an energy source, including but not limited to ultrasound, radiofrequency or laser, from the outer housing to ablate or alter the pain fibers in the annulus, preferably within the one-third outer layer of disc annulus. The energy source may be an ablation catheter inserted into the infusion or suction port of the tissue disrupting apparatus, or inserted to the target site after removal of the tissue disrupting apparatus.

Some embodiments may comprise methods for treating and/or monitoring the status of an intervertebral disc by measuring and/or monitoring pressure in the intervertebral disc. The monitoring may be independent of, before, during and/or after a disc treatment procedure, for example, in order to achieve a safe and successful patient outcome. The devices and methods described herein may be used in conjunction with surgical procedures, wherein at least a portion of a disc nucleus is removed, or otherwise modified in order to benefit the spinal column, for example, to effect decompression of an intervertebral disc, for example, a herniated disc.

It is known that an intervertebral disc nucleus has an intrinsic pressure. In the event the disc pressure becomes elevated, for example, due to injury or trauma, the disc itself may bulge, or nucleus material from the center of the disc may extrude through fissures in the annulus and impinge on nearby nerves, causing severe pain and physical disability. As described elsewhere herein, various surgical techniques are known which are directed at reducing the extent to which an intervertebral disc presses against nearby nerve structures. In some embodiments, methods may optionally include determining an initial disc pressure prior to such a surgical technique and a post surgery disc pressure, for example, a pressure within a desired range. Some embodiments may also include methods for monitoring the intrinsic pressure in the disc nucleus during a surgical procedure. One example includes a surgical procedure directed at reducing disc size or disc pressure. Some embodiments may utilize aspiration alone, or in conjunction with cutting, chopping, grinding, emulsification or ablation to reduce the volume of nucleus material within the disc. The use of enzymes or other therapeutic agents suitable for dissolving or breaking down the nucleus material or reducing disc pressure may be employed as part of a procedure. In some embodiments, methods for monitoring a patient may include measuring the intrinsic pressure within an intervertebral disc nucleus before, during and/or after medical treatment of the disc. The monitoring may be performed intermittently, periodically, or on a substantially continuous real-time basis. In some embodiments, the method allows a physician to utilize the pressure information obtained from the disc in diagnosing a problem, determining potential or actual effectiveness of a treatment, and/or determining the degree of treatment necessary to achieve a desired result. Treatment may occur during the diagnostic procedure or at a later visit. Same day treatment may be performed using the same or a different access pathway to the disc.

### Biopsy Procedures

Although non-invasive methods for examining tissue, such as manual palpation, X-ray, MRI, CT, and ultrasound imaging, are often used in the initial work-up of a medical problem, the diagnosis and treatment of patients with tumors, pre-malignant conditions, infectious lesions and nodules, rheumatologic disorders and other disorders often utilize tissue biopsies to confirm the diagnosis. When a healthcare provider suspects that an organ or tissue may contain cancerous or diseased cells or tissues, a biopsy may be performed, using either an open procedure or a percutaneous procedure. For an open procedure, a scalpel is used by the surgeon to create a large incision in the tissue, in order to provide direct viewing and to access the tissue mass of interest. Removal of the entire mass (excisional biopsy) or a part of the mass (incisional biopsy) may be done.

For a percutaneous biopsy, a needle or cannula-like instrument is used with a small incision to access the tissue mass of interest and to obtain a tissue sample for later examination and analysis. The potential advantages of the percutaneous method as compared to the open method include less recovery time for the patient, less pain, shorter surgical and anesthesia time, lower cost, less risk of injury to adjacent bodily tissues such as nerves, and less disfigurement of the patient's anatomy. Percutaneous biopsies, however, are subject to sampling errors that may increase the rate of false-negative results, and may still cause inadvertent injury and bleeding to adjacent body structures. For this reason, percutaneous procedures are sometimes combined with artificial imaging modalities, such as X-ray and ultrasound, to improve the reliability of diagnoses and treatments.

Percutaneous sampling methods may include aspiration and core sampling. Aspiration of the tissue through a fine needle often requires that the target tissue be fragmented into small enough pieces to be withdrawn through the fine needle in a fluid medium. The method is less intrusive than other known sampling techniques, but may be limited to examination of isolated cells or small cell clumps in the liquid (cytology), rather than the cells and the tissue structure (histology). In core biopsy, a core or fragment of tissue is obtained for histologic examination, which may be done via a frozen or paraffin section. This type of biopsy is may be more invasive, with an increased risk of bleeding and associated with a less desirable cosmetic result. The type of biopsy used may depend on the suspected disease and various factors present in the patient.

In some embodiments, where a single intact tissue specimen is desired, a tissue disrupting apparatus with one or more cutting edges may be used. Instead of rotating the cutting edge with a motor as described in other embodiments, the cutting may be manually rotated or manipulated to cause a single piece of tissue to be removed. In other embodiments, the cutting edge may be vibrated or reciprocated to facilitate cutting. In some embodiments, after the cutting procedure, the tissue sample may be retained in the tissue disrupting apparatus for removal from the body. In some embodiments, retaining the tissue sample may be performed by proximally withdrawing the cutting edge to trap the tissue sample within a lumen of the tissue disrupting apparatus, or by collapsing the cutting edge to clamp or trap the tissue sample. In some embodiments, tissue sampling without high-speed rotation of a tissue disrupting element may be preferred where a risk of spreading malignant cells is present.

It is to be understood that this invention is not limited to particular exemplary embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “a blade” includes a plurality of such blades and reference to “the energy source” includes reference to one or more sources of energy and equivalents thereof known to those skilled in the art, and so forth.

The publications discussed herein are provided solely for their disclosure. Nothing herein is to be construed as an admission that the present invention is not entitled to antedate such publication by virtue of prior invention. Further, the dates of publication provided, if any, may be different from the actual publication dates which may need to be independently confirmed.

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims. For all the embodiments described herein, the steps of the method need not be performed sequentially.

What is claimed as new and desired to be protected by Letters Patent of the United States is:

1. A device for removing material from a body, comprising:
a drive shaft comprising a proximal section, a distal section and a longitudinal shaft axis therebetween;
a motor coupled to the proximal section of the drive shaft; and
at least one tissue disrupting member comprising a proximal section and a distal section and having a collapsed configuration and a deployed configuration;
wherein the proximal section of the tissue disrupting member is coupled to the distal section of the drive shaft at a proximal coupling zone, and wherein the collapsed configuration of the tissue disrupting member exerts greater bending stress on the proximal end of the tissue disrupting member than the deployed configuration.

2. The device of claim 1, wherein the tissue disrupting member is preshaped to its deployed configuration.

3. The device of claim 1, wherein the proximal section of the disrupting member is integral with the distal section of the drive shaft.

4. The device of claim 1, wherein the deployed configuration of at least one tissue disrupting member comprises a bend that is distal to the proximal coupling zone.

5. The device of claim 4, wherein the bend is at least about 1 mm distal to the coupling zone.

6. The device of claim 5, wherein the bend is at least about 1.5 mm distal to the coupling zone.

7. The device of claim 6, wherein the bend is at least about 2 mm distal to the coupling zone.

8. The device of claim 4, wherein the device comprises at least two tissue disrupting members and at least one slot between at least two tissue disrupting members, and wherein the at least one slot comprises a proximal slot end and a distal slot end.

9. The device of claim 8, wherein the proximal slot end of at least one slot is longitudinally located between the proximal coupling zone and the bend of at least one tissue disrupting member.

10. The device of claim 9, wherein the distal slot end of at least one slot is longitudinally located distal to the bend of at least one tissue disrupting member.

11. The device of claim 4, wherein the tissue disrupting member proximal to the bend comprises a generally straight configuration.

12. The device of claim 1, wherein the tissue disrupting member is an elongate disrupting member.

13. The device of claim 12, wherein the distal section of the elongate tissue disrupting member is coupled to a slide member that is slidably located in a lumen of the drive shaft.

14. The device of claim 1, further comprising a helical transport structure.

15. The device of claim 14, wherein the helical structure is integral with a surface of the drive shaft.

16. The device of claim 14, wherein the helical structure is independently movable from the drive shaft.

17. The device of claim 1, further comprising a housing with a motor cavity, a drive shaft aperture, a drive shaft lumen between the motor cavity and the drive shaft aperture, a tubing connector and a lumen between the drive shaft lumen and the tubing connector, and a motor controller.

18. The device of claim 17, wherein the motor controller is configured to permit user-controlled movement of the drive shaft in two or more directions.

19. The device of claim 13, further comprising a slide controller configured to permit user-controlled movement of the slide member with respect to the drive shaft.

20. The device of claim 1, wherein the distal end of at least one tissue disrupting member comprises a free distal end.

21. The device of claim 4, wherein at least one tissue disrupting member slidably resides in a distal lumen of the distal section of the drive shaft.

22. The device of claim 21, wherein at least one tissue disrupting member comprises an elongate wire, polymer or fiber structure.

23. The device of claim 20, wherein at least one tissue disrupting member comprises a plate member.

24. The device of claim 23, wherein the plate member is a non-planar plate member.

25. The device of claim 23, wherein the proximal end of the plate member comprises a flange configuration.

26. The device of claim 1, wherein an outermost portion of the tissue disrupting member is located about 1 mm to about 5 mm from the longitudinal axis of the drive shaft in the collapsed configuration and about 2 mm to 13 mm in the deployed position.

27. The device of claim 1, wherein at least one tissue disrupting member comprises a material selected from a group consisting of nickel-titanium alloy, stainless steel, cobalt-chromium alloy, nickel-cobalt-chromium-molybdenum alloy, and titanium-aluminum-vanadium alloy.

28. The device of claim 1, further comprising from about three tissue disrupting members to about six tissue disrupting members.

29. A method of removing tissue, comprising:
providing a tissue disrupting device comprising a drive shaft and a plurality of tissue disrupting members coupled to the drive shaft at a coupling zone;
exerting a greater stress on the plurality of non-linear tissue disrupting members at a distal stress zone that is distal to the coupling zone and a lesser stress at a proximal stress zone located between the coupling zone and the distal stress zone to restrain the tissue disrupting device;
inserting the restrained tissue disrupting device into a body;
positioning the restrained tissue disrupting device about a target area in the body;
reducing the greater stress at the distal stress zone of the plurality of non-linear tissue disrupting members; and
actuating the plurality of tissue disrupting members to disrupt tissue at the target area.

30. The method of claim 29, wherein actuating the plurality of tissue disrupting member comprises rotating the plurality of disrupting members at a speed of about 5,000 rpm to about 100,000 rpm.

31. The method of claim 29, wherein actuating the plurality of tissue disrupting member comprises rotating the plurality of disrupting members at a speed of about 3,000 rpm to about 20,000 rpm.

32. The method of claim 29, further comprising emulsifying tissue at the target area.

33. The method of claim 29, further comprising rotating an auger to transport disrupted tissue away from the target area.

34. The method of claim 33, wherein the plurality of tissue disrupting members and the auger are rotated independently.

35. The method of claim 29, further comprising:
applying suction to the target area to transport disrupted tissue away from the target area.

36. The method of claim 29, further comprising:
adjusting the greater stress at the distal stress zone to modify at least one dimension of the plurality of tissue disrupting members.

37. The method of claim 29, further comprising:
adjusting the greater stress at the distal stress zone to reduce at least one dimension of the plurality of tissue disrupting members;
repositioning the tissue disrupting device so that the plurality of tissue disrupting members to a second target area;
readjusting the greater stress at the distal stress zone to increase at least one dimension of the plurality of tissue disrupting members; and
rotating the strip portion to disrupt tissue at the second target area.

38. A method of manufacturing a disrupting device, comprising:
providing a tubular body comprising a proximal end, a distal end, and a midsection therebetween;
creating a plurality of struts with disrupting edges in the midsection of the tubular body by forming a plurality of slots between the proximal and distal ends of the tubular body;
shaping the midsection of the tubular body in a radially outward direction without straining the tubular body by more than 8%;
heat annealing the tubular body to reduce the strain;
reshaping the heat annealed midsection in a radially outward direction without straining the tubular body by more than 8%; and
heat annealing the reshaped tubular body to reduce the strain.

39. The method of claim 38, further comprising coupling the tubular body to a motor with a rotatable shaft.
